SEQUENCE LISTING (12 sequences)

SEQ ID NO: 1 - protein, 5 aa, Artificial Sequence
  Description of Artificial Sequence Heavy chain variable region complementary determining region CDR1 (murine and humanized)
SYYIH

SEQ ID NO: 2 - protein, 17 aa, Artificial Sequence
  Description of Artificial Sequence Heavy chain variable region complementary determining region CDR2 (murine and humanized)
  MISC_FEATURE (16)..(16): Xaa is Lys or Gln
VIYPGNDDISYNQKFXG

SEQ ID NO: 3 - protein, 9 aa, Artificial Sequence
  Description of Artificial Sequence Heavy chain variable region complementary determining region CDR3 (murine and humanized)
EVRLRYFDV

SEQ ID NO: 4 - protein, 17 aa, Artificial Sequence
  Description of Artificial Sequence Light chain variable region complementary determining region CDR1 (murine and humanized)
KSSQSVFFSSSQKNYLA

SEQ ID NO: 5 - protein, 7 aa, Artificial Sequence
  Description of Artificial Sequence Light chain variable region complementary determining region CDR2 (murine and humanized)
WASTRES

SEQ ID NO: 6 - protein, 8 aa, Artificial Sequence
  Description of Artificial Sequence Light chain variable region complementary determining region CDR3 (murine and humanized)
HQYLSSRT

SEQ ID NO: 7 - protein, 118 aa, Artificial Sequence
  Description of Artificial Sequence Murine heavy chain variable region
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 8 - protein, 113 aa, Artificial Sequence
  Description of Artificial Sequence Murine light chain variable region
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 9 - protein, 118 aa, Artificial Sequence
  Description of Artificial Sequence Humanized heavy chain variable region
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 10 - protein, 113 aa, Artificial Sequence
  Description of Artificial Sequence Humanized light chain variable region
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 11 - protein, 447 aa, Artificial Sequence
  Description of Artificial Sequence Full length humanized heavy chain region
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 219 aa, Artificial Sequence
  Description of Artificial Sequence Full length humanized light chain region
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0160089 A1
(43) Pub. Date: May 30, 2019

(54) COMBINATION TREATMENT WITH ANTIBODY-DRUG CONJUGATES AND CYTARABINE

(71) Applicant: IMMUNOGEN, INC., Waltham, MA (US)

(72) Inventors: Sharlene Adams, Waltham, MA (US); Callum M. Sloss, Wakefield, MA (US); Patrick Zweidler-McKay, Lincoln, MA (US)

(21) Appl. No.: 16/175,165

(22) Filed: Oct. 30, 2018

Related U.S. Application Data
(60) Provisional application No. 62/586,973, filed on Nov. 16, 2017.
(60) Provisional application No. 62/579,184, filed on Oct. 31, 2017.

(51) Int. Cl. A61K 31/7068; A61P 35/02; A61K 31/5517; C07K 16/28; A61K 47/68

(52) U.S. Cl. CPC A61K 31/7068; A61P 35/02; A61K 47/6803; C07K 16/2803; A61K 31/5517

(57) ABSTRACT

The present invention provides a method of treating a cancer in a subject comprising administering to the subject an effective amount of a CD33-targeted antibody-drug conjugate (ADC) and an effective amount of cytarabine. Also provided are pharmaceutical compositions comprising an effective amount of a CD33-targeted ADC and an effective amount of cytarabine.

### CROSS REFERENCE TO RELATED APPLICATIONS

This application claims the benefit of the filing dates, under 35 U.S.C. § 119(e), of U.S. Provisional Application No. 62/579,184, filed Oct. 31, 2017 and U.S. Provisional Application No. 62/586,973, filed Nov. 16, 2017. The entire contents of each of the above-referenced applications are incorporated herein by reference.

### TECHNICAL FIELD

The present invention provides a method of treating a cancer in a subject comprising administering to the subject an effective amount of a CD33-targeted antibody-drug conjugate (ADC) and an effective amount of cytarabine. Also provided are pharmaceutical compositions comprising an effective amount of a CD33-targeted ADC and an effective amount of cytarabine.

### BACKGROUND OF THE INVENTION

Acute myeloid leukemia (AML) is associated with the accumulation of abnormal blast cells in bone marrow. Acute myeloid leukemia (AML) is one of the most common types of leukemia among adults. In the United States alone, over 18,000 new cases of AML are identified each year, and more than 10,000 deaths are associated with AML. Despite high initial response rates to chemotherapy, many acute myeloid leukemia (AML) patients fail to achieve complete remission. In fact, the majority of patients with AML relapse within 3-5 years from diagnosis.

The leukocyte differentiation antigen CD33 is a 364 amino acid transmembrane glycoprotein with sequence homology to members of the sialoadhesin family, including myelin-associated glycoprotein and CD22, as well as sialoadhesin itself (S. Peiper, 2002, Leucocyte Typing VII, White Cell Differentiation, Antigens, Proceedings of the Seventh International Workshop and Conference, Oxford University Press, p. 777).

Expression of CD33 appears to be highly specific to the hematopoietic compartment, with strong expression by myeloid precursor cells (S. Peiper, 2002). It is expressed by myeloid progenitor cells such as CFU-GEMM, CFU-GM, CFU-G and BFU-E, monocytes/macrophages, granulocyte precursors such as promyelocytes and myelocytes although with decreased expression upon maturation and differentiation, and mature granulocytes though with a low level of expression (S. Peiper, 2002). Anti-CD33 monoclonal antibodies have shown that CD33 is expressed by clonogenic, acute myelogenous leukemia (AML) cells in greater than 80% of human cases (LaRussa, V. F. et al., 1992, Exp. Hematol. 20:442-448). In contrast, pluripotent hematopoietic stem cells that give rise to “blast colonies” in vitro (Leary, A. G. et al., 1987, Blood 69:953) and that induce hematopoietic long-term marrow cultures (Andrews R. G. et al., 1989, J. Exp. Med. 169:1721; Sutherland, H. J. et al., 1989, Blood 74:1563) appear to lack expression of CD33.

Due to the selective expression of CD33, antibody drug conjugates (hereinafter “ADCs”) that combine cytotoxic drugs with monoclonal antibodies that specifically recognize and bind CD33 have been proposed for use in selective targeting of AML cells. Such therapies are expected to leave stem cells and primitive hematopoietic progenitors unaffected. Recently, a CD33-targeted ADC utilizing a novel DNA alkylator, DGN462, which comprises an indolino-benzodiazepine dimer containing a monoimine moiety has been reported (see, for example, U.S. Pat. Nos. 8,765,740, 8,889,669, 9,169,272, and 9,434,748) that shows anti-cancer activity in in vitro and in vivo hematologic cancer models. While this ADC shows great promise, there is still a need for improved methods for using the ADC in treating patients suffering from cancer, in particular hematologic cancers, such as, AML.

### SUMMARY OF THE INVENTION

It has now been surprisingly found that the combination of cytarabine with a CD33-targeted ADC containing an indolino-benzodiazepine dimer cytotoxic payload has synergistic effects against leukemia cells both in vitro and in vivo as compared with the ADC alone and cytarabine alone (see Examples 1-4). In addition, strong synergies between the indolino-benzodiazepine cytotoxic payload and Chk1/2 inhibitors and between the indolino-benzodiazepine cytotoxic payload and Mdm2 inhibitors were also observed in 12 human AML cell lines (Example 5).

Cytarabine, also known as cytosine arabinoside (ara-C), is a chemotherapy medication used to treat acute myeloid leukemia (AML), acute lymphocytic leukemia (ALL), chronic myelogenous leukemia (CML), and non-Hodgkin's lymphoma.

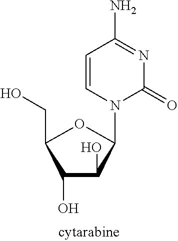

In a first embodiment, the present invention provides a method for treating a cancer in a subject comprising administering to the subject an effective amount of cytarabine and an effective amount of an ADC of Formula (I):

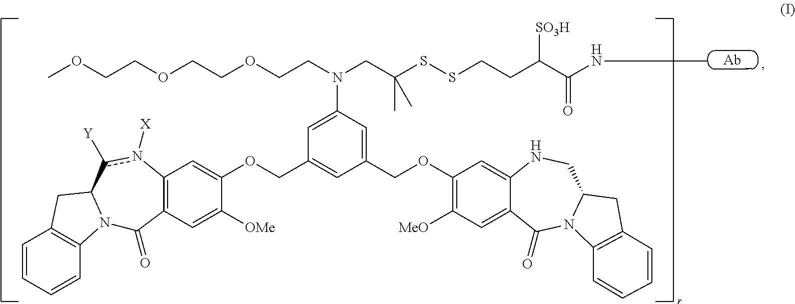

or a pharmaceutically acceptable salt thereof. The double line [symbol] between N and C represents either a single bond or a double bond, provided that when it is a double bond, X is absent and Y is hydrogen; and when it is a single bond, X is hydrogen and Y is —SO₃H. The term “Ab” is an anti-CD33 antibody or antigen-binding fragment thereof. Alternatively, “Ab” is an anti-CD33 antibody or antigen-binding fragment comprising a heavy chain variable region (V_{H}) complementary determining region (CDR)1 sequence of SEQ ID NO:1, a V_{H} CDR2 sequence of SEQ ID NO:2, and a V_{H} CDR3 sequence of SEQ ID NO:3, and a light chain variable region (V_{L}) CDR1 sequence of SEQ ID NO:4, a V_{L} CDR2 sequence of SEQ ID NO:5, and a V_{L} CDR3 sequence of SEQ ID NO:6. The term “r” is an integer from 1 to 10.

Also provided in the first embodiment of the invention is an ADC of Formula (I) or a pharmaceutically acceptable salt thereof for treating a subject with cancer, in combination with cytarabine.

The first embodiment of the invention also provides the use of an ADC of Formula (I) or a pharmaceutically acceptable salt thereof for the manufacture of a medicament for treating a subject with cancer in combination with cytarabine.

In one embodiment, cytarabine is administered to the subject prior to the administration of the ADC. In another embodiment, cytarabine and the ADC are administered to the subject concurrently.

In one embodiment, a total daily dose of 20-3000 mg/m² of cytarabine is administered to the subject. In another embodiment, cytarabine is administered to the subject daily. In another embodiment, cytarabine is administered to the subject every other day.

In one embodiment, a total daily dose of 110 mg/m² of cytarabine is administered to the subject every day for 7 days.

In another embodiment, a total daily dose of 3000 mg/m² of cytarabine is administered to the subject every other day for 5 days.

In another embodiment, a total daily dose of 20 mg/m² of cytarabine is administered to the subject every day for 10 days.

In another embodiment, a total daily dose of 200 mg/m² of cytarabine is administered to the subject every day for 7 days.

In one embodiment, the subject is treated with cytarabine and the antibody-drug conjugate for 5 days, a week, 10 days, 2 weeks, 3 weeks or 1 month.

In one embodiment, the combination of cytarabine and the ADC is used as a front line therapy for treating AML in a fit AML patient. In another embodiment, the combination of cytarabine and the ADC is used as a front line therapy for treating AML in an unfit AML patient.

In one embodiment, the combination of cytarabine and the ADC is used as a second line therapy for treating AML in a fit AML patient. In another embodiment, the combination of cytarabine and the ADC is used as a second line therapy for treating AML in an unfit AML patient.

In one embodiment, the combination of cytarabine and the ADC is used as a second line therapy for treating refractory or relapse AML in a fit AML patient. In another embodiment, the combination of cytarabine and the ADC is used as a second line therapy for treating refractory or relapse AML in an unfit AML patient.

In a second embodiment, the present invention provides a method of treating a cancer in a subject comprising administering to the subject an effective amount of a Chk1/2 inhibitor and an effective amount of an ADC of Formula (I) described in the first embodiment.

Also provided in the second embodiment of the invention is an ADC of Formula (I) or a pharmaceutically acceptable salt thereof for treating a subject with cancer, in combination with a Chk1/2 inhibitor.

The second embodiment of the invention also provides the use of an ADC of Formula (I) or a pharmaceutically acceptable salt thereof for the manufacture of a medicament for treating a subject with cancer in combination with a Chk1/2 inhibitor.

In a third embodiment, the present invention provides a method of treating a cancer in a subject comprising administering to the subject an effective amount of a Mdm2 inhibitor and an effective amount of an ADC of Formula (I) described in the first embodiment.

Also provided in the third embodiment of the invention is an ADC of Formula (I) or a pharmaceutically acceptable salt thereof for treating a subject with cancer, in combination with a Mdm2 inhibitor.

The third embodiment of the invention also provides the use of an ADC of Formula (I) or a pharmaceutically acceptable salt thereof for the manufacture of a medicament for treating a subject with cancer in combination with a Mdm2 inhibitor.

In a fourth embodiment, the present invention provides a method of treating a cancer in a subject comprising administering to the subject an effective amount of cytarabine, an effective amount of a Chk1/2 inhibitor and an effective amount of an ADC of Formula (I) described in the first embodiment.

Also provided in the fourth embodiment of the invention is an ADC of Formula (I) or a pharmaceutically acceptable salt thereof for treating a subject with cancer, in combination with cytarabine and a Chk1/2 inhibitor.

The fourth embodiment of the invention also provides the use of an ADC of Formula (I) or a pharmaceutically acceptable salt thereof for the manufacture of a medicament for treating a subject with cancer in combination with cytarabine and a Chk1/2 inhibitor.

In a fifth embodiment, the present invention provides a method of treating a cancer in a subject comprising administering to the subject an effective amount of cytarabine, an effective amount of a Mdm2 inhibitor and an effective amount of an ADC of Formula (I) described in the first embodiment.

Also provided in the fifth embodiment of the invention is an ADC of Formula (I) or a pharmaceutically acceptable salt thereof for treating a subject with cancer, in combination with cytarabine and a Mdm2 inhibitor.

The fifth embodiment of the invention also provides the use of an ADC of Formula (I) or a pharmaceutically acceptable salt thereof for the manufacture of a medicament for treating a subject with cancer in combination with cytarabine and a Mdm2 inhibitor.

In a sixth embodiment, the present invention provides a method of treating a cancer in a subject comprising administering to the subject an effective amount of cytarabine, an effective amount of a Chk1/2 inhibitor, an effective amount of a Mdm2 inhibitor and an effective amount of an ADC of Formula (I) described in the first embodiment.

Also provided in the sixth embodiment of the invention is an ADC of Formula (I) or a pharmaceutically acceptable salt thereof for treating a subject with cancer, in combination with cytarabine, a Chk1/2 inhibitor and a Mdm2 inhibitor.

The sixth embodiment of the invention also provides the use of an ADC of Formula (I) or a pharmaceutically acceptable salt thereof for the manufacture of a medicament for treating a subject with cancer in combination with cytarabine, a Chk1/2 inhibitor and Mdm2 inhibitor.

In certain embodiments, for the methods described in the first, second, third, fourth, fifth or sixth embodiments, the method further comprises administering to the subject an effective amount of an additional chemotherapeutic agent.

In certain embodiments, for the methods described herein (e.g., the methods of the first, second or third embodiment), the cancer is selected from the group consisting of acute myeloid leukemia (AML), chronic myeloid leukemia (CML), acute lymphoblastic leukemia (ALL), B-cell lineage acute lymphoblastic leukemia (B ALL), T-cell lineage acute lymphoblastic leukemia (T-ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, eosinophilic leukemia, B myelomonocytic leukemia and Hodgkin's leukemia (HL). In one embodiment, the cancer is chemotherapy sensitive. In another embodiment, the cancer is chemotherapy resistant. In yet another embodiment, the cancer is acute myeloid leukemia (AML). In yet another embodiment, the AML is refractory or relapse acute myeloid leukemia. In one embodiment, the subject being treated with the methods described herein is a fit AML subject. In another embodiment, the subject being treated with the methods described herein is an unfit AML subject. In yet another embodiment, the AML is characterized by overexpression of P-glycoprotein, overexpression of EVIL a p53 alteration, DNMT3A mutation, FLT3 internal tandem duplication, a complex karyotype, decreased expression in BRCA1, BRCA2, or PALB2, or mutations in BRCA1, BRCA2, or PALB2.

In certain embodiments, for the methods described herein (e.g., the methods of the first, second or third embodiment), the anti-CD33 antibody or antigen-binding fragment thereof comprises a heavy chain variable region comprising an amino acid sequence having at least 95% identity to the amino acid sequence of SEQ ID NO:7 or 9. In one embodiment, the anti-CD33 antibody or antigen-binding fragment thereof comprises a light chain variable region comprising an amino acid sequence having at least 95% identity to the amino acid sequence of SEQ ID NO:8 or 10. In another embodiment, the antibody or antigen-binding fragment thereof comprises a heavy chain variable region comprising the sequence of SEQ ID NO:9 and a light chain variable region comprising the sequence of SEQ ID NO:10. In another embodiment, the anti-CD33 antibody comprises a heavy chain having the amino acid sequence set forth in SEQ ID NO:11 and a light chain having the amino acid sequence set forth in SEQ ID NO:12. In another embodiment, the antibody is huMy9-6. In yet another embodiment, the antibody is a CDR-grafted or resurfaced antibody.

ADC1, ADC2, IMGN779 (defined below) and pharmaceutically acceptable salts thereof, are specific examples of ADCs that can be used in the disclosed methods of treatment (e.g., the methods of the first, second or third embodiment).

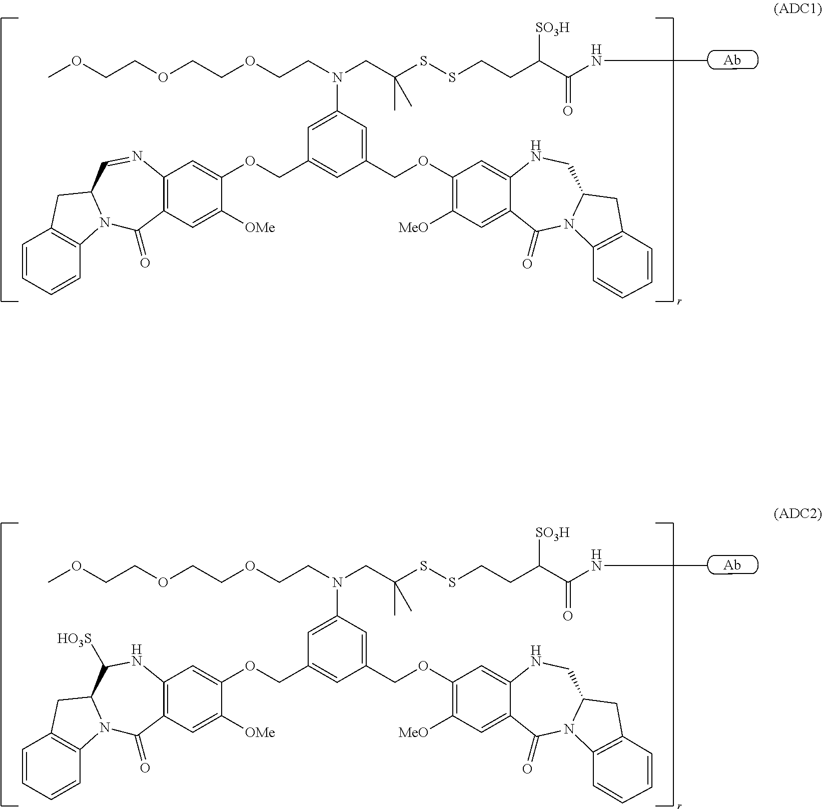

“Ab” is as defined for Formula (I). The term “r” is an integer from 1 to 10. Methods of preparing ADC1, ADC2, and IMGN779 are provided in U.S. Pat. Nos. 8,765,740 and 9,353,127, the entire teachings of which are incorporated herein by reference.

Pharmaceutically acceptable salts are those which are suitable for use in humans and animals without undue toxicity, irritation, and allergic response. Examples for suitable salts for the ADC of Formula (I), ADC1, ADC2, and IMGN779 are disclosed in U.S. Pat. No. 8,765,740, the entire teachings of which are incorporated herein by reference. In one embodiment, the pharmaceutically acceptable salt for the ADCs of Formula (I), ADC1, ADC2, and IMGN779 is the sodium or potassium salt.

In certain embodiments, the ADC of Formula (I) is represented by the following formula:

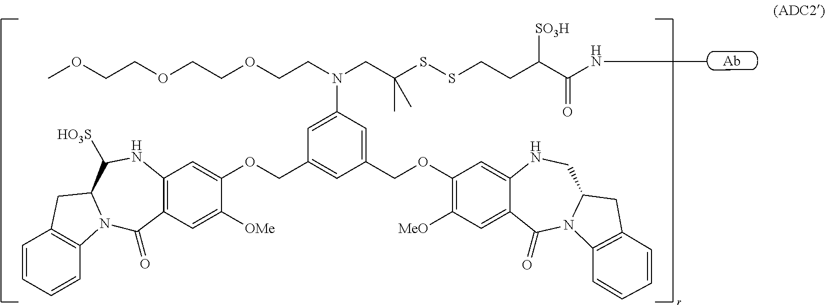

A fourth embodiment of the invention is a pharmaceutical composition comprising: i) an effective amount of cytarabine; ii) an effective amount of an ADC of Formula (I), ADC1, ADC2, or IMGN779 or a pharmaceutically acceptable salt thereof; and iii) a pharmaceutically acceptable carrier or diluent. In one embodiment, the pharmaceutically acceptable salt for the ADCs of Formula (I), ADC1, ADC2, and IMGN779 is the sodium or potassium salt. In another embodiment, the ADC of Formula (I) is ADC2′.

In a fifth embodiment, the present invention provides a pharmaceutical composition comprising: i) an effective amount of a Chk1/2 inhibitor; ii) an effective amount of an ADC of Formula (I), ADC1, ADC2, ADC2′ or IMGN779 or a pharmaceutically acceptable salt thereof; and iii) a pharmaceutically acceptable carrier or diluent. In one embodiment, the pharmaceutically acceptable salt for the ADCs of Formula (I), ADC1, ADC2, and IMGN779 is the sodium or potassium salt. In another embodiment, the ADC of Formula (I) is ADC2′.

In a sixth embodiment, the present invention provides a pharmaceutical composition comprising: i) an effective amount of a Mdm2 inhibitor; ii) an effective amount of an ADC of Formula (I), ADC1, ADC2, or IMGN779 or a pharmaceutically acceptable salt thereof; and iii) a pharmaceutically acceptable carrier or diluent. In one embodiment, the pharmaceutically acceptable salt for the ADCs of Formula (I), ADC1, ADC2, and IMGN779 is the sodium or potassium salt. In another embodiment, the ADC of Formula (I) is ADC2′.

### BRIEF DESCRIPTION OF THE DRAWINGS

FIG. 1 shows in vivo efficacy of the combination of IMGN779 (single dose) and cytarabine in EOL-1 subcutaneous model.

FIG. 2 shows in vivo efficacy of the combination of IMGN779 (QW×3) and cytarabine in MV4-11 disseminated model.

FIG. 3 shows in vivo efficacy of the combination of IMGN779 (QW×3) and cytarabine in Molm-13 disseminated model.

FIGS. 4A and 4B show in vitro evaluation of CD33 expression in MV4-11 (FIG. 4A) and MOLM-13 (FIG. 4B) cells post-treatment with cytarabine.

FIGS. 5A and 5B show in vitro apoptotic response to the combination treatment of IMGN779 and cytarabine in MV4-11 (FIG. 5A) and MOLM-13 (FIG. 5B) cells.

FIG. 6 shows strong synergy between IGN free payload and Chk1/2 inhibitors and Mdm2 inhibitors in 12 human AML cell lines.

FIG. 7 shows cell cycle analysis carried out by flow cytometry measuring the fluorescence of Prodium Iodide added to methanol-fixed in MV4-11 AML cell line.

FIG. 8 shows in vitro evaluation of p-CHK2 and p21 expression in cells treated with IMGN779 alone, cytarabine alone, or the combination of IMGN779 and cytarbine.

FIG. 9 shows in vitro evaluation of cPARP, cCaspase 3, and Annexin V expression in cells treated with IMGN779 alone, cytarabine alone, or the combination of IMGN779 and cytarabine.

### DETAILED DESCRIPTION OF THE INVENTION

The present invention features methods of treating patients with cancers, e.g., a hematologic cancer, such as AML, by administering a combination of a CD33-targeted ADC containing an indolino-benzodiazepine dimer cytotoxic payload, in particular, the ADC of Formula (I), and cytarabine.

The invention is based, at least in part, on the discovery that the combination of cytarabine and IMGN779 is more active in vitro against AML cells and in vivo against AML xenografts in mice than the individual agents alone. IMGN779, a CD33-targeted antibody drug conjugate comprising an anti-huCD33 antibody, huMy9-6 antibody (also termed as Z4681A antibody), conjugated to a novel DNA-alkylating agent, DGN462, via a cleavable disulfide linker. In certain instance, synergistic effect was observed for the combination of cytarabine and IMGN779 (see e.g., Table 3 of Example 1). In addition, it is surprisingly found that administration of cytarabine can increase expression of CD33 in human AML cell lines, and may potentiate the CD33-dependent uptake of IMGN779 by AML cells, and consequently the efficacy of IMGN779 against AML cells.

### Definitions

“IMGN779” is a CD33-targeted ADC comprising the huMy9-6 antibody, also termed as Z4681A antibody (i.e., an antibody comprising the heavy chain CDR1-3 having the sequence of SEQ ID NOs:1-3, respectively and the light chain CDR1-3 having the sequence of SEQ ID NOs:4-6; an antibody comprising the heavy chain variable region having the sequence of SEQ ID NO:9 and a light chain variable region having the sequence of SEQ ID NO:10; or an antibody comprising the heavy chain sequence having the sequence of SEQ ID NO:11 and the light chain sequence having the sequence of SEQ ID NO:12), conjugated to DGN462, via a cleavable disulfide linker. IMGN779 may be represented as ADC3 as depicted below:

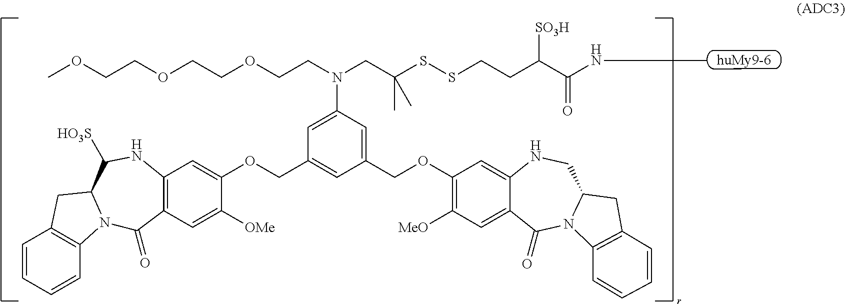

or a pharmaceutically acceptable salt thereof, wherein the average value for r in a composition comprising ADC3 is between 2.4 and 3.0; or IMGN779 may also be represented as ADC4 as depicted below:

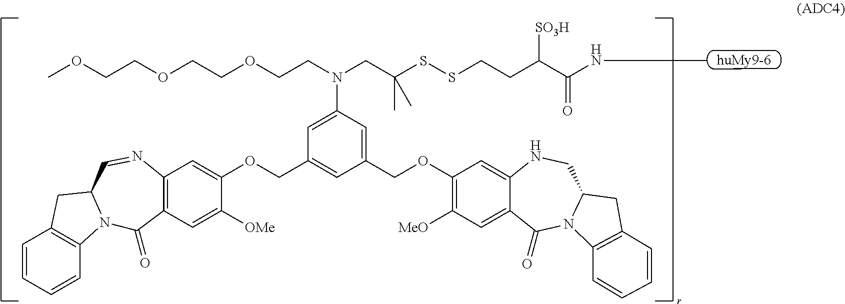

or a pharmaceutically acceptable salt thereof, wherein the average value for r in a composition comprising ADC4 is between 2.4 and 3.0; or IMGN779 can be a combination of ADC3 and ADC4 or pharmaceutically acceptable salts thereof.

In certain embodiments, IMGN779 is a sodium salt of ADC3 and is represented by the following formula, wherein the average value for r in a composition comprising sodium salt of ADC3 is between 2.4 and 3.0:

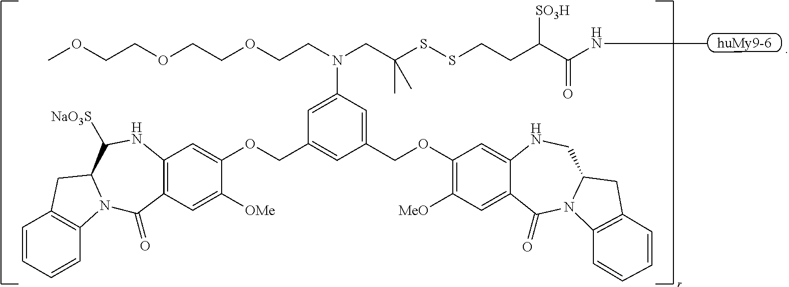

By “P-glycoprotein” is meant a polypeptide or fragment thereof having at least about 85% amino acid sequence identity to the human sequence provided at NCBI Accession No. NP_001035830 and conferring multi-drug resistance on a cell in which it is expressed. The sequence of an exemplary human P-glycoprotein is provided below:

| | |
|---|---|
| 1 | maaaeaggdd arcvrlsaer aqalladvdt llfdcdgvlw rgetavpgap ealralrarg |
| 61 | krlgfitnns sktraayaek lrrlgfggpa gpgaslevfg tayctalylr qrlagapapk |
| 121 | ayvlgspala aeleavgvas vgvgpeplqg egpgdwlhap lepdvravvv gfdphfsymk |
| 181 | ltkalrylqq pgcllvgtnm dnrlplengr fiagtgclvr avemaaqrqa diigkpsrfi |
| 241 | fdcvsqeygi npertvmvgd rldtdillga tcglktiltl tgvstlgdvk nnqesdcvsk |
| 301 | kkmvpdfyvd siadllpalq g |

By “CD33 protein” is meant a polypeptide or fragment thereof having at least about 85% amino acid sequence identity to the human sequence provided at NCBI Accession No. CAD36509 and having anti-CD33 antibody binding activity. An exemplary human CD33 amino acid sequence is provided below:

| | |
|---|---|
| 1 | mplllllpll wagalamdpn fwlqvqesvt vqeglcvlvp ctffhpipyy dknspvhgyw |
| 61 | fregaiisrd spvatnkldq evqeetqgrf rllgdpsrnn cslsivdarr rdngsyffrm |
| 121 | ergstkysyk spqlsvhvtd lthrpkilip gtlepghskn ltcsyswace qgtppifswl |
| 181 | saaptslgpr tthssvliit prpqdhgtnl tcqvkfagag vttertiqln vtyvpqnptt |
| 241 | gifpgdgsgk qetragvvhg aiggagvtal lalclcliff ivkthrrkaa rtavgrndth |
| 301 | pttgsaspkh qkksklhgpt etsscsgaap tvemdeelhy aslnfhgmnp skdtsteyse |
| 361 | vrtq |

By “FLT3 protein,” “FLT3 polypeptide,” “FLT3,” “FLT-3 Receptor,” or “FLT-3R” is meant a polypeptide or fragment thereof having at least about 85%, 90%, 95%, 99% or 100% amino acid sequence identity to the human sequence of FLT3 tyrosine kinase receptor, also referred to as FLK-2 and STK-1, provided at NCBI Accession No. NP_004110 and having tyrosine kinase activity, including receptor tyrosine kinase activity. In one embodiment the FLT3 amino acid sequence is the human FLT3 amino acid sequence provided below:

| | |
|---|---|
| 1 | mpalardggq lpllvvfsam ifgtitnqdl pvikcvlinh knndssvgks ssypmvsesp |
| 61 | edlgcalrpq ssgtvyeaaa vevdvsasit lqvlvdapgn isclwvfkhs slncqphfdl |
| 121 | qnrgvvsmvi lkmtetqage yllfiqseat nytilftvsi rntllytlrr pyfrkmenqd |
| 181 | alvcisesvp epivewvlcd sqgesckees pavvkkeekv lhelfgtdir ccarnelgre |
| 241 | ctrlftidln qtpqttlpql flkvgeplwi rckavhvnhg fgltwelenk aleegnyfem |
| 301 | stystnrtmi rilfafvssv arndtgyytc ssskhpsqsa lvtivekgfi natnssedye |
| 361 | idqyeefcfs vrfkaypqir ctwtfsrksf pceqkgldng ysiskfcnhk hqpgeyifha |
| 421 | enddaqftkm ftlnirrkpq vlaeasasqa scfsdgyplp swtwkkcsdk spncteeite |
| 481 | gvwnrkanrk vfgqwvssst lnmseaikgf lvkccaynsl gtscetilln spgpfpfiqd |
| 541 | nisfyatigv cllfivvltl lichkykkqf ryesqlqmvq vtgssdneyf yvdfreyeyd |
| 601 | lkwefprenl efgkvlgsga fgkvmnatay gisktgvsiq vavkmlkeka dsserealms |
| 661 | elkmmtqlgs henivnllga ctlsgpiyli feyccygdll nylrskrekf hrtwteifke |
| 721 | hnfsfyptfq shpnssmpgs revqihpdsd qisglhgnsf hsedeieyen qkrieeeedl |
| 781 | nvltfedllc fayqvakgme flefkscvhr dlaarnvlvt hgkvvkicdf glardimsds |
| 841 | nyvvrgnarl pvkwmapesl fegiytiksd vwsygillwe ifslgvnpyp gipvdanfyk |
| 901 | liqngfkmdq pfyateeiyi imqscwafds rkrpsfpnlt sflgcqlada eeamyqnvdg |
| 961 | rvsecphtyq nrrpfsremd lgllspqaqv eds |

By “FLT3-ITD” is meant a FLT3 polypeptide having internal tandem duplication(s) including but not limited to simple tandem duplication(s) and/or tandem duplication(s) with insertion. In various embodiments, FLT3 polypeptides having internal tandem duplications are activated FLT3 variants (e.g., constitutively autophosphorylated). In some embodiments, the FLT3-ITD includes tandem duplications and/or tandem duplication(s) with insertion in any exon or intron including, for example, exon 11, exon 11 to intron 11, and exon 12, exon 14, exon 14 to intron 14, and exon 15. The internal tandem duplication mutation (FLT3-ITD) is the most common FLT3 mutation, present in about 20-25% of AML cases. Patients with FLT3-ITD AML have a worse prognosis than those with wild-type (WT) FLT3, with an increased rate of relapse and a shorter duration of response to chemotherapy.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between 6⁻³ and 6⁻¹⁰⁰ indicating a closely related sequence.

An “anti-CD33 antibody or antigen-binding fragment thereof” refers to an antibody or antigen-binding fragment thereof that specifically binds to CD33.

By “specifically binds” is meant an antibody or fragment thereof that recognizes and binds a polypeptide of interest, but which does not substantially recognize and bind other molecules in a sample, for example, a biological sample, which naturally includes a polypeptide of the invention.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

“Effective amount” means that amount of ADC, cytarabine, the Chk1/2 inhibitor, or the Mdm2 inhibitor that elicits the desired biological response in a subject. Such response includes alleviation of the symptoms of the disease or disorder being treated, inhibition or a delay in the recurrence of symptom of the disease or of the disease itself, an increase in the longevity of the subject compared with the absence of the treatment, or inhibition or delay in the progression of symptom of the disease or of the disease itself. Toxicity and therapeutic efficacy of the ADC, cytarabine, the Chk1/2 inhibitor, or the Mdm2 inhibitor can be determined by standard pharmaceutical procedures in cell cultures and in experimental animals. The effective amount of the ADC, cytarabine, the Chk1/2 inhibitor, or the Mdm2 inhibitor to be administered to a subject will depend on the stage, category and status of the multiple myeloma and characteristics of the subject, such as general health, age, sex, body weight and drug tolerance. The effective amount of the ADC, cytarabine, the Chk1/2 inhibitor, or the Mdm2 inhibitor to be administered will also depend on administration route and dosage form. Dosage amount and interval can be adjusted individually to provide plasma levels of the active compound that are sufficient to maintain desired therapeutic effects.

The terms “treatment,” “treat,” and “treating” refer to reversing, alleviating, or inhibiting the progress of a cancer, or one or more symptoms thereof, as described herein.

The terms “administer”, “administering”, “administration”, and the like, as used herein, refer to methods that may be used to enable delivery of the ADCs, cytarabine, the Chk1/2 inhibitor, and the Mdm2 inhibitor to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. In one aspect, the ADC, cytarabine, the Chk1/2 inhibitor, and/or the Mdm2 inhibitor are administered intravenously.

By “fit AML” is meant a subject having AML who is eligible for intensive therapy. The measures for determining a subject with fit AML includes, e.g., physical performance (as determined by e.g., the Eastern Cooperative Oncology Group performance status (ECOG PS), the Karnofsky performance status (KPS), and the short physical performance battery (SPPB)), comorbid conditions (as determined by the Charlson comorbidity index (CCI) or the hematopoietic cell transplantation-specific comorbidity index (HCT-CI)), cognitive function, or prognostic models (including but not limited to, cytogenetic group, age, white blood cell count, LDH, type of AML). In some cases, a fit AML subject is a subject under the age of 60.

By “unfit AML” is meant a subject having AML who is ineligible for intensive therapy. The measures for determining a subject with unfit AML includes, e.g., physical performance (as determined by e.g., the Eastern Cooperative Oncology Group performance status (ECOG PS), the Karnofsky performance status (KPS), and the short physical performance battery (SPPB)), comorbid conditions (as determined by the Charlson comorbidity index (CCI) or the hematopoietic cell transplantation-specific comorbidity index (HCT-CI)), cognitive function, or prognostic models (including but not limited to, cytogenetic group, age, white blood cell count, LDH, type of AML). In some cases, an unfit AML subject is a subject over the age of 60.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Anti-CD33 Antibodies

In one embodiment, the antibody in the ADC of formula (I), ADC1, ADC2 or ADC2′ is an anti-CD33 antibody, in particular, huMy9-6 antibody.

“My9-6”, “murine My9-6” and “muMy9-6”, is the murine anti-CD33 antibody from which huMy9-6 is derived. My9-6 is fully characterized with respect to the germline amino acid sequence of both light and heavy chain variable regions, amino acid sequences of both light and heavy chain variable regions, the identification of the CDRs, the identification of surface amino acids and means for its expression in recombinant form. See, for example, U.S. Pat. Nos. 7,557,189; 7,342,110; 8,119,787; 8,337,855 and U.S. Patent Publication No. 20120244171, each of which is incorporated herein by reference in their entirety. The amino acid sequences of muMy9-6 are also shown below in Table 1. The My9-6 antibody has also been functionally characterized and shown to bind with high affinity to CD33 on the surface of CD33-positive cells.

The term “variable region” is used herein to describe certain portions of antibody heavy chains and light chains that differ in sequence among antibodies and that cooperate in the binding and specificity of each particular antibody for its antigen. Variability is not usually evenly distributed throughout antibody variable regions. It is typically concentrated within three segments of a variable region called complementarity-determining regions (CDRs) or hypervariable regions, both in the light chain and the heavy chain variable regions. The more highly conserved portions of the variable regions are called the framework regions. The variable regions of heavy and light chains comprise four framework regions, largely adopting a beta-sheet configuration, with each framework region connected by the three CDRs, which form loops connecting the beta-sheet structure, and in some cases forming part of the beta-sheet structure. The CDRs in each chain are held in close proximity by the framework regions and, with the CDRs from the other chain, contribute to the formation of the antigen binding site of antibodies (E. A. Kabat et al. Sequences of Proteins of Immunological Interest, Fifth Edition, 1991, NIH). The “constant” region is not involved directly in binding an antibody to an antigen, but exhibits various effector functions, such as participation of the antibody in antibody-dependent cellular toxicity.

TABLE 1

| | Amino acid sequence |
|---|---|
| Heavy chain | SYYIH |
| variable region | (SEQ ID NO: 1) |
| complementary | |
| determining | |
| region CDR1 | |
| (murine and | |
| humanized) | |
| Heavy chain | VIYPGNDDISYNQKFXG |
| variable region | (SEQ ID NO: 2), |
| complementary | wherein X is K or Q |
| determining | |
| region CDR2 | |
| (murine and | |
| humanized) | |
| Heavy chain | EVRLRYFDV |
| variable region | (SEQ ID NO: 3) |
| complementary | |
| determining | |
| region CDR3 | |
| (murine and | |
| humanized) | |
| Light chain | KSSQSVFFSSSQKNYLA |
| variable region | (SEQ ID NO: 4) |
| complementary | |
| determining | |
| region CDR1 | |
| (murine and | |
| humanized) | |
| Light chain | WASTRES |
| variable region | (SEQ ID NO: 5) |
| complementary | |
| determining | |
| region CDR2 | |
| (murine and | |
| humanized) | |
| Light chain | HQYLSSRT |
| variable region | (SEQ ID NO: 6) |
| complementary | |
| determining | |
| region CDR3 | |
| (murine and | |
| humanized) | |
| Murine heavy | QVQLQQPGAEVVKPGASVKMSCKASGYTFT |
| chain variable | SYYIHWIKQTPGQGLEWVGVIYPGNDDISY |
| region | NQKFKGKATLTADKSSTTAYMQLSSLTSED |
| | SAVYYCAREVRLRYFDVWGAGTTVTVSS |
| | (SEQ ID NO: 7) |
| Murine light | NIMLTQSPSSLAVSAGEKVTMSCKSSQSVF |
| chain variable | FSSSQKNYLAWYQQIPGQSPKLLIYWASTR |
| region | ESGVPDRFTGSGSGTDFTLTISSVQSEDLA |
| | IYYCHQY LSSRTFGGGTKLEIKR |
| | (SEQ ID NO: 8) |

Humanized versions of My9-6, variously designated herein as “huMy9-6”, and “humanized My9-6”, are also described.

The goal of humanization is a reduction in the immunogenicity of a xenogenic antibody, such as a murine antibody, for introduction into a human, while maintaining the full antigen binding affinity and specificity of the antibody. Humanized antibodies may be produced using several technologies, such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641 (Pedersen et al.), which is hereby incorporated in its entirety by reference. Briefly, in a preferred method, (1) position alignments of a pool of antibody heavy and light chain variable regions are generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 angstroms of any atom of any residue of the complementarity-determining regions of the rodent antibody; and (5) the humanized rodent antibody having binding specificity is produced.

Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5):489-498; Studnicka G. M. et al., 1994, Protein Engineering 7(6):805-814; Roguska M. A. et al., 1994, PNAS 91:969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

As further described herein, the CDRs of My9-6 were identified by modeling and their molecular structures were predicted. Humanized My9-6 antibodies were then prepared and have been fully characterized as described, for example in U.S. Pat. Nos. 7,342,110 and 7,557,189, which are incorporated herein by reference. The amino acid sequences of the light and heavy chains of a number of huMy9-6 antibodies are described, for example, in U.S. Pat. Nos. 8,337,855 and 8,765,740, each of which is incorporated herein by reference. The amino acid sequences shown in Table 2 describe the huMy9-6 antibody of the invention.

TABLE 2

| | Amino acid sequence |
|---|---|
| Humanized | QVQLQQPGAEVVKPGASVKMSCKASGYTFTSYY |
| heavy chain | IHWIKQTPGQGLEWVGVIYPGNDDISYNQKFQG |
| variable | KATLTADKSSTTAYMQLSSLTSEDSAVYY |
| region | CAREVRLRYFDVWGQGTTVTVSS |
| | (SEQ ID NO: 9) |
| Humanized | EIVLTQSPGSLAVSPGERVTMSCKSSQSVFFSS |
| light chain | SQKNYLAWYQQIPGQSPRLLIYWASTRESGVPD |
| variable | RFTGSGSGTDFTLTISSVQPEDLAIYYCHQYLS |
| region | SRTFGQGTKLEIKR |
| | (SEQ ID NO: 10) |
| Full length | QVQLQQPGAEVVKPGASVKMSCKASGYTFTSYY |
| humanized | IHWIKQTPGQGLEWVGVIYPGNDDISYNQKFQG |
| heavy chain | KATLTADKSSTTAYMQLSSLTSEDSAVYYCARE |
| region | VRLRYFDVWGQGTTVTVSSASTKGPSVFPLAPS |
| | SKSTSGGTAALGCLVKDYFPEPVTVSWNSGALT |
| | SGVHTFPAVLQSSGLYSLSSVVTVPSSSLGTQT |
| | YICNVNHKPSNTKVDKKVEPKSCDKTHTCPPCP |
| | APELLGGPSVFLFPPKPKDTLMISRTPEVTCVV |
| | VDVSHEDPEVKFNWYVDGVEVHNAKTKPREEQY |
| | NSTYRVVSVLTVLHQDWLNGKEYKCKVSNKALP |
| | APIEKTISKAKGQPREPQVYTLPPSRDELTKNQ |
| | VSLTCLVKGFYPSDIAVEWESNGQPENNYKTTP |
| | PVLDSDGSFFLYSKLTVDKSRWQQGNVFSCSVM |
| | HEALHNHYTQKSLSLSPG |
| | (SEQ ID NO: 11) |
| Full length | EIVLTQSPGSLAVSPGERVTMSCKSSQSVFFSS |
| humanized | SQKNYLAWYQQIPGQSPRLLIYWASTRESGVPD |
| light chain | RFTGSGSGTDFTLTISSVQPEDLAIYYCHQYLS |
| region | SRTFGQGTKLEIKRTVAAPSVFIFPPSDEQLKS |
| | GTASVVCLLNNFYPREAKVQWKVDNALQSGNSQ |
| | ESVTEQDSKDSTYSLSSTLTLSKADYEKHKVYA |
| | CEVTHQGLSSPVTKSFNRGEC |
| | (SEQ ID NO: 12) |

Although epitope-binding fragments of the murine My9-6 antibody and the humanized My9-6 antibodies are discussed herein separately from the murine My9-6 antibody and the humanized versions thereof, it is understood that the term “antibody” or “antibodies” of the present invention may include both the full length muMy9-6 and huMy9-6 antibodies as well as epitope-binding fragments of these antibodies.

In a further embodiment, there are provided antibodies or epitope-binding fragments thereof comprising at least one complementarity-determining region having an amino acid sequence selected from the group consisting of SEQ ID NOs:1-6, and having the ability to bind CD33.

In a further embodiment, there are provided antibodies or epitope-binding fragments thereof comprising at least one heavy chain variable region and at least one light chain variable region, wherein said heavy chain variable region comprises three complementarity-determining regions having amino acid sequences represented by SEQ ID NOs:1-3, respectively, and wherein said light chain variable region comprises three complementarity-determining regions having amino acid sequences represented by SEQ ID NOs:4-6, respectively.

In a further embodiment, there are provided antibodies having a heavy chain variable region that has an amino acid sequence that shares at least 90% sequence identity with an amino acid sequence represented by SEQ ID NO:7, more preferably 95% sequence identity with SEQ ID NO:7, most preferably 100% sequence identity with SEQ ID NO:7.

Similarly, there are provided antibodies having a light chain variable region that has an amino acid sequence that shares at least 90% sequence identity with an amino acid sequence represented by SEQ ID NO:8, more preferably 95% sequence identity with SEQ ID NO:8, most preferably 100% sequence identity with SEQ ID NO:8.

In a further embodiment, antibodies are provided having a humanized (e.g., resurfaced, CDR-grafted) heavy chain variable region that shares at least 90% sequence identity with an amino acid sequence represented by SEQ ID NO:9, more preferably 95% sequence identity with SEQ ID NO:9, most preferably 100% sequence identity with SEQ ID NO:9.

Similarly, antibodies are provided having a humanized (e.g., resurfaced, CDR-grafted) light chain variable region that shares at least 90% sequence identity with an amino acid sequence corresponding to SEQ ID NO:10, more preferably 95% sequence identity with SEQ ID NO:10, most preferably 100% sequence identity with SEQ ID NO:10. In particular embodiments, the antibody includes conservative mutations in the framework region outside of the CDRs.

As used herein, “antibody fragments” include any portion of an antibody that retains the ability to bind to CD33, generally termed “epitope-binding fragments.” Examples of antibody fragments preferably include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Epitope-binding fragments, including single-chain antibodies, may comprise the variable region(s) alone or in combination with the entirety or a portion of the following: hinge region, C_{H1}, C_{H2}, and C_{H3} domains. Such fragments may contain one or both Fab fragments or the F(ab′)₂ fragment. Preferably, the antibody fragments contain all six CDRs of the whole antibody, although fragments containing fewer than all of such regions, such as three, four or five CDRs, are also functional. Further, the functional equivalents may be or may combine members of any one of the following immunoglobulin classes: IgG, IgM, IgA, IgD, or IgE, and the subclasses thereof. Fab and F(ab′)₂ fragments may be produced by proteolytic cleavage, using enzymes such as papain (Fab fragments) or pepsin (F(ab′)₂ fragments). The single-chain FVs (scFvs) fragments are epitope-binding fragments that contain at least one fragment of an antibody heavy chain variable region (V_{H}) linked to at least one fragment of an antibody light chain variable region (V_{L}). The linker may be a short, flexible peptide selected to assure that the proper three-dimensional folding of the (V_{L}) and (V_{H}) regions occurs once they are linked so as to maintain the target molecule binding-specificity of the whole antibody from which the single-chain antibody fragment is derived. The carboxyl terminus of the (V_{L}) or (V_{H}) sequence may be covalently linked by a linker to the amino acid terminus of a complementary (V_{L}) and (V_{H}) sequence. Single-chain antibody fragments may be generated by molecular cloning, antibody phage display library or similar techniques well known to the skilled artisan. These proteins may be produced, for example, in eukaryotic cells or prokaryotic cells, including bacteria.

The epitope-binding fragments of the present invention can also be generated using various phage display methods known in the art. In phage display methods, functional antibody domains are displayed on the surface of phage particles which carry the polynucleotide sequences encoding them. In particular, such phage can be utilized to display epitope-binding domains expressed from a repertoire or combinatorial antibody library (e.g., human or murine). Phage expressing an epitope-binding domain that binds the antigen of interest can be selected or identified with antigen, e.g., using labeled CD33 or CD33 bound or captured to a solid surface or bead. Phage used in these methods are typically filamentous phage including Fd and M13 binding domains expressed from phage with Fab, Fv or disulfide-stabilized Fv antibody domains recombinantly fused to either the phage gene III or gene VIII protein.

Examples of phage display methods that can be used to make the epitope-binding fragments of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

After phage selection, the regions of the phage encoding the fragments can be isolated and used to generate the epitope-binding fragments through expression in a chosen host, including mammalian cells, insect cells, plant cells, yeast, and bacteria, using recombinant DNA technology, e.g., as described in detail below. For example, techniques to recombinantly produce Fab, Fab′ and F(ab′)₂ fragments can also be employed using methods known in the art such as those disclosed in PCT publication WO 92/22324; Mullinax et al., 1992, BioTechniques 12(6):864-869; Sawai et al., 1995, AJRI34:26-34; and Better et al., 1988, Science 240:1041-1043; said references incorporated by reference in their entireties. Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; Skerra et al., 1988, Science 240:1038-1040.

Also included within the scope of the invention are functional equivalents of the My9-6 antibody and the humanized My9-6 antibodies. The term “functional equivalents” includes antibodies with homologous sequences, chimeric antibodies, modified antibody and artificial antibodies, for example, wherein each functional equivalent is defined by its ability to bind to CD33. The skilled artisan will understand that there is an overlap in the group of molecules termed “antibody fragments” and the group termed “functional equivalents.”

Antibodies with homologous sequences are those antibodies with amino acid sequences that have sequence identity or homology with amino acid sequence of the murine My9-6 and humanized My9-6 antibodies of the present invention. Preferably identity is with the amino acid sequence of the variable regions of the murine My9-6 and humanized My9-6 antibodies of the present invention. “Sequence identity” and “sequence homology” as applied to an amino acid sequence herein is defined as a sequence with at least about 90%, 91%, 92%, 93%, or 94% sequence identity, and more preferably at least about 95%, 96%, 97%, 98%, or 99% sequence identity to another amino acid sequence, as determined, for example, by the FASTA search method in accordance with Pearson and Lipman, Proc. Natl. Acad. Sci. USA 85, 2444-2448 (1988).

As used herein, a chimeric antibody is one in which different portions of an antibody are derived from different animal species. For example, an antibody having a variable region derived from a murine monoclonal antibody paired with a human immunoglobulin constant region. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entireties.

The CDRs are of primary importance for epitope recognition and antibody binding. However, changes may be made to the residues that comprise the CDRs without interfering with the ability of the antibody to recognize and bind its cognate epitope. For example, changes that do not affect epitope recognition, yet increase the binding affinity of the antibody for the epitope may be made.

Thus, also included in the scope of the present invention are improved versions of both the murine and humanized antibodies, which also specifically recognize and bind CD33, preferably with increased affinity.

Several studies have surveyed the effects of introducing one or more amino acid changes at various positions in the sequence of an antibody, based on the knowledge of the primary antibody sequence and on its properties such as binding and level of expression (Yang, W. P. et al., 1995, J. Mol. Biol., 254, 392-403; Rader, C. et al., 1998, Proc. Natl. Acad. Sci. USA, 95, 8910-8915; Vaughan, T. J. et al., 1998, Nature Biotechnology, 16, 535-539).

In these studies, equivalents of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J. et al., 1998, Nature Biotechnology, 16, 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (Gram, H. et al., 1992, Proc. Natl. Acad. Sci. USA, 89, 3576-3580; Boder, E. T. et al., 2000, Proc. Natl. Acad. Sci. USA, 97, 10701-10705; Davies, J. and Riechmann, L., 1996, Immunotechnology, 2, 169-179; Thompson, J. et al., 1996, J. Mol. Biol., 256, 77-88; Short, M. K. et al., 2002, J. Biol. Chem., 277, 16365-16370; Furukawa, K. et al., 2001, J. Biol. Chem., 276, 27622-27628).

By a similar directed strategy of changing one or more amino acid residues of the antibody, the antibody sequences described herein (e.g., in Tables 1 and 2) can be used to develop anti-CD33 antibodies with improved functions, including improved affinity for CD33. Improved antibodies also include those antibodies having improved characteristics that are prepared by the standard techniques of animal immunization, hybridoma formation and selection for antibodies with specific characteristics.

### CD33-Targeted Antibody Drug Conjugates

In certain embodiments, the present invention provides a method of treating a cancer, e.g., a hematologic cancer, in a subject comprising administering to the subject an effective amount of cytarabine and an effective amount of an ADC of Formula (I):

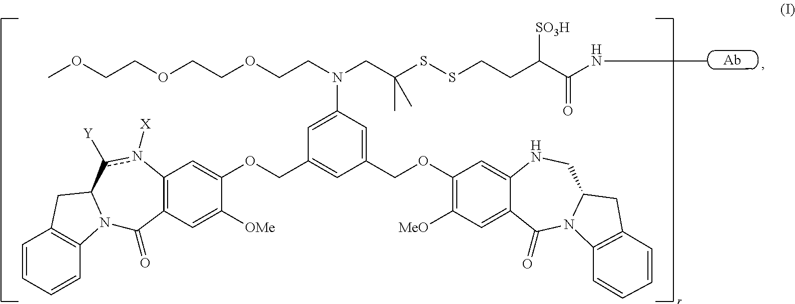

or a pharmaceutically acceptable salt thereof. The double line [symbol] between N and C represents either a single bond or a double bond, provided that when it is a double bond, X is absent and Y is hydrogen; and when it is a single bond, X is hydrogen and Y is —SO₃H. The term “Ab” is an anti-CD33 antibody or antigen-binding fragment thereof comprising a heavy chain variable region (V_{H}) complementary determining region (CDR)1 sequence of SEQ ID NO:1, a V_{H} CDR2 sequence of SEQ ID NO:2, and a V_{H} CDR3 sequence of SEQ ID NO:3, and a light chain variable region (V_{L}) CDR1 sequence of SEQ ID NO:4, a V_{L} CDR2 sequence of SEQ ID NO:5, and a V_{L} CDR3 sequence of SEQ ID NO:6. The term “r” is an integer from 1 to 10.

ADC1, ADC2, ADC2′, IMGN779, and pharmaceutically acceptable salts thereof, are specific examples of ADCs that can be used in the disclosed methods of treatment.

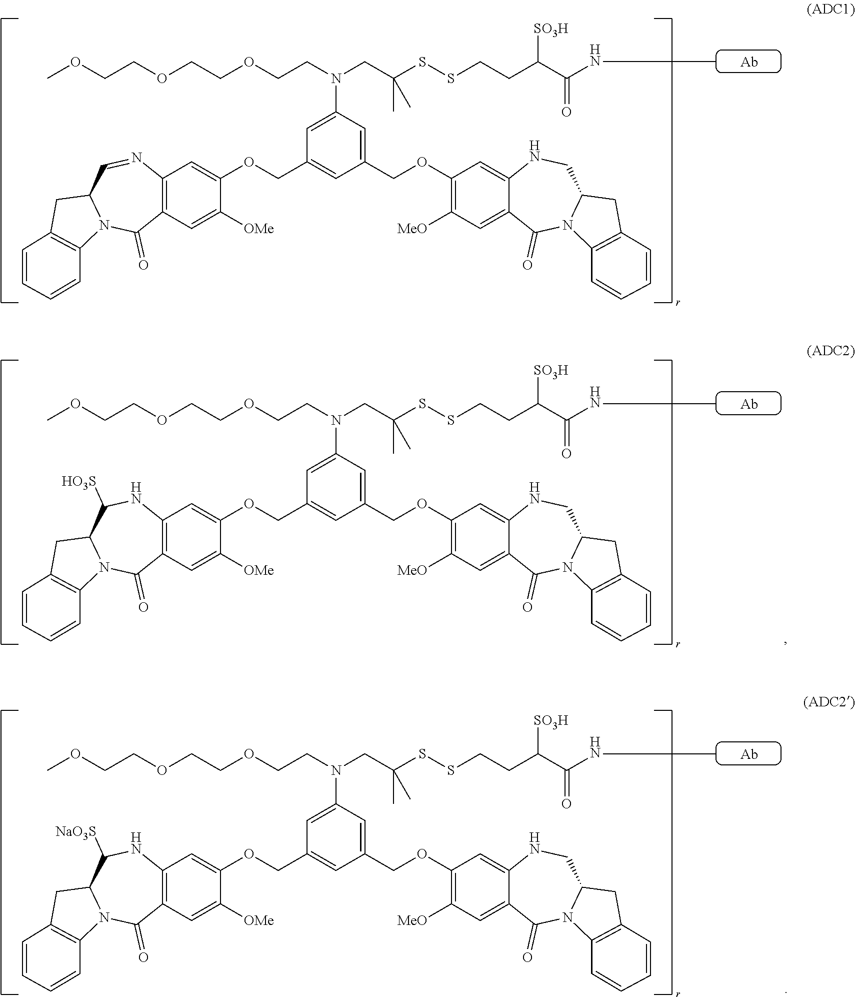

“Ab” is as defined for Formula (I). The term “r” is an integer from 1 to 10. Methods of preparing ADC1, ADC2, ADC2′ and IMGN779 are provided in U.S. Pat. Nos. 8,765,740 and 9,353,127, the entire teachings of which are incorporated herein by reference.

In some embodiments, for ADC of formula (I), ADC1, ADC2 or ADC2′ or a pharmaceutically acceptable salt thereof, the anti-CD33 antibody or antigen-binding fragment thereof comprises a heavy chain variable region comprising an amino acid sequence having at least 95% identity to the amino acid sequence of SEQ ID NO:7 or 9. In another embodiment, the anti-CD33 antibody or antigen-binding fragment thereof comprises a light chain variable region comprising an amino acid sequence having at least 95% identity to the amino acid sequence of SEQ ID NO:8 or 10.

In other embodiments, the antibody portion of the ADC of formula (I), ADC1, ADC2 or ADC2′ is an anti-CD33 antibody comprising a heavy chain variable region having at least about 90%, 91%, 92%, 93%, or 94% sequence identity, and more preferably at least about 95%, 96%, 97%, 98%, or 99% sequence identity to SEQ ID NO:9 and a light chain variable region having at least about 90%, 91%, 92%, 93%, or 94% sequence identity, and more preferably at least about 95%, 96%, 97%, 98%, or 99% sequence identity to SEQ ID NO:10.

In some embodiments, the antibody portion of the ADC of formula (I), ADC1, ADC2 or ADC2′ comprises a heavy chain variable region comprising the sequence of SEQ ID NO:9 and a light chain variable region comprising the sequence of SEQ ID NO:10. In some embodiments, the antibody portion of the ADC of formula (I), ADC1, ADC2 or ADC2′ is an anti-CD33 antibody comprising a heavy chain having the amino acid sequence set forth in SEQ ID NO:11 and a light chain having the amino acid sequence set forth in SEQ ID NO:12.

In some embodiments, the antibody portion of the ADC of formula (I), ADC1, ADC2 or ADC2′ is the huMy9-6 antibody, also termed as “Z4681A.” In one embodiment, the antibody is a CDR-grafted or resurfaced antibody.

In specific embodiments, the CD33-targeted ADC is IMGN779. IMGN779 comprises the huMy9-6 antibody, also termed as Z4681A antibody, conjugated to DGN462, via a cleavable disulfide linker. IMGN779 may be represented as depicted below as ADC3:

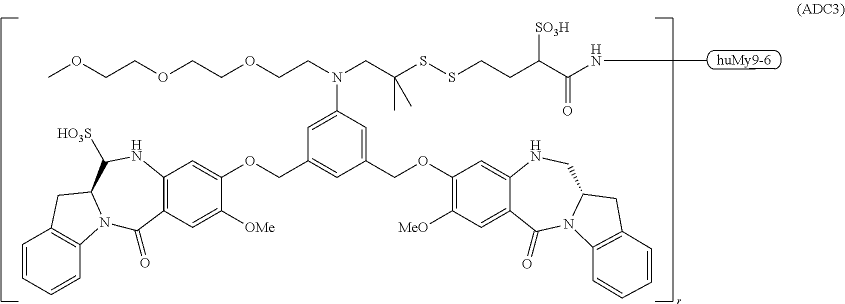

or a pharmaceutically acceptable salt thereof (e.g., sodium salt); or IMGN779 may also be represented below as ADC4:

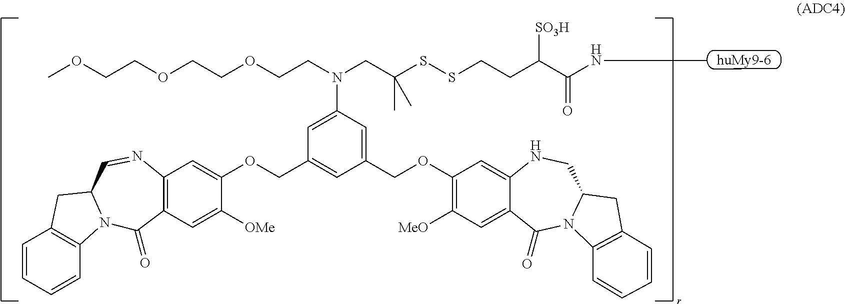

or a pharmaceutically acceptable salt thereof; or IMGN may also be a combination of ADC3 and ADC4.

In certain embodiments, the conjugate described herein may comprise 1-10 cytotoxic benzodiazepine dimer compounds, 2-9 cytototoxic benzodiazepine dimer compounds, 3-8 cytotoxic benzodiazepine dimer compounds, 4-7 cytotoxic benzodiazepine dimer compounds, or 5-6 cytotoxic benzodiazepine dimer compounds.

In certain embodiments, a composition comprising the conjugates described herein may comprise an average of 1-10 cytotoxic benzodiazepine dimer molecule per antibody molecule. The average ratio of cytotoxic benzodiazepine dimer molecule per antibody molecule is referred to herein as the Drug Antibody Ratio (DAR). In one embodiment, the DAR is between 2-8, 3-7, 3-5, 2.5-3.5 or 2.4-3.0. In one embodiment, the DAR is 2.4, 2.5, 2.6, 2.7, 2.8, 2.9, 3.0, 3.1, 3.2, 3.3, 3.4 or 3.5. In another embodiment, the DAR is 2.8.

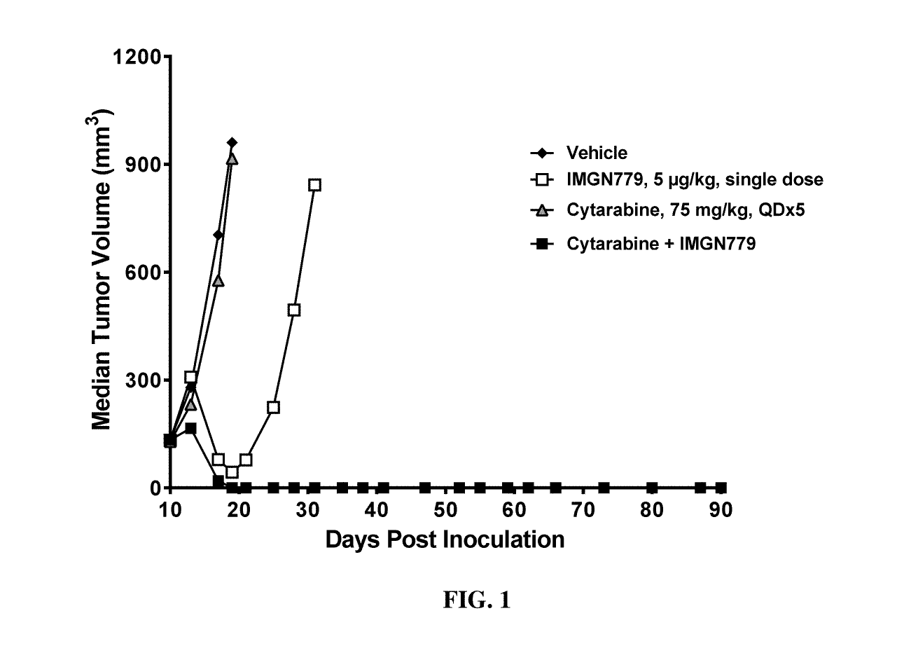

The cytotoxic benzodiazepine dimer compound and the conjugates described herein can be prepared according to methods described in U.S. Pat. Nos. 8,765,740 and 9,353,127, for example, but not limited to, paragraphs M3951403971 and M5981406071, FIGS. 1, 15, 22, 23, 38-41, 43, 48, 55 and 60, and Examples 1, 6, 12, 13, 20, 21, 22, 23, 26-30 and 32 of U.S. Pat. No. 8,765,740 and paragraphs M0071401051, M1971402911, FIGS. 1-11, 16, 28 and Examples 1-7, 9-13, 15 and 16 of U.S. Pat. No. 9,353,127.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). Preferably, the cation is monovalent.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. In particular embodiments, the pharmaceutically acceptable salt is a sodium or a potassium salt.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

### Cytarabine

Cytarabine, also known as cytosine arabinoside (ara-C), is a chemotherapeutic agent used to treat acute myeloid leukemia (AML), acute lymphocytic leukemia (ALL), chronic myelogenous leukemia (CML), and non-Hodgkin's lymphoma. It is given by injection into a vein (i.e. intravenous or IV), under the skin, or into the cerebrospinal fluid.

In certain embodiments, for the methods of the present invention described herein, cytarabine can be administered to the subject in a total daily dose of 20-3000 mg/m², 20-50 mg/m², 50-200 mg/m², 200-500 mg/m², 500-1000 mg/m², or 1000-3000 mg/m².

As used herein, a “total daily dose” in the context of cytarabine refers to the total amount of cytarabine that can be administered to the subject within one day.

In certain embodiments, for the methods of the present invention described herein, cytarabine can be administered to the subject daily or every other day. In certain embodiments, cytarabine can be administered for 5 days, 6 days, a week, 8 days, 9 days, 10 days, 2 weeks, 3 weeks, 4 weeks, 2 months, 3 months, etc.

In certain embodiments, for the methods of the present invention described herein, cytarabine can be administered to the subject in a total daily dose of 110 mg/m² every day for 7 days (e.g., on days 1-7 of the treatment regimen).

In certain embodiments, a high dose (e.g., 3,000 mg/m² total daily dose) of cytarabine can be administered to the subject every other day (e.g., on days 1, 3 and 5 of the treatment regimen).

In certain embodiments, a low dose (e.g., 20 mg/m² or 30 mg/m² total daily dose) of cytarabine can be administered to the subject every day for 10 days, for example, on days 1-10 of the treatment regimen. In one embodiment, the low dose is 20 mg/m² total daily dose. In another embodiment, the low dose is 30 mg/m² total daily dose.

In certain embodiments, an intermediate dose (e.g., 200 mg/m² total daily dose) of cytarabine cam be administered to the subject every day for 7 days, for example on days 1-7 of the treatment regimen.

### Chk1/2 and Mdm2 Inhibitors

Checkpoint kinase 1 (Chk1), also known as Chek1, is an serine/threonine-specific protein kinase. It is encoded by the CHEK1 gene, located on chromosome 11 (11q22-23) in humans Chk1 coordinates the DNA damage response (DDR) and cell cycle checkpoint response. Activation of Chk1 results in the initiation of cell cycle checkpoints, cell cycle arrest, DNA repair and cell death to prevent damaged cells from progressing through the cell cycle.

Checkpoint kinase 2 (Chk2), also known as Chek2, is a serine threonine kinase and is encoded by the tumor suppressor gene CHEK2, located on the long (q) arm of chromosome 22. Chk2 operates in a complex network of proteins to elicit DNA repair, cell cycle arrest or apoptosis in response to DNA damage. Mutations to the CHEK2 gene have been linked to a wide range of cancers including breast cancer.

As used herein, a “Chk1/2 inhibitor” refers to substances that decrease Chk1 and/or Chk2 activities.

Suitable Chk1 inhibitors that can be used in the present invention include, but are not limited to, UCN-01 (7-hydroxystaruosporine), MK-8776, AZD7762, PF-477736, LY2603618, LY2606368, SCH90076, AZD7762, XL844 and CHR0124. Other suitable Chk1 inhibitors are those described in Velic, D. et al., Biomolecules, 2015, 5, 3204-3259; Garrett, M. D. et al., Trends Pharmacol. Sci. 2011, 32, 308-316; and Prudhomme, M., Recent Pat. Anticancer Drug Discov. 2006, 1, 55-68, each of which is incorporated herein by reference in its entirety.

Suitable Chk2 inhibitors that can be used in the present invention include, but are not limited to, PV1019 ([7-nitro-1H-indole-2-caryboxylic acid {(4-[1-guanidinohydrazone)-ethyl]-phenyl}-amide]), CCT241533, BML277/C3742 (2-(4-(4-chlorophenoxy)phenyl)-1H-benzimidazole-5-carboxamide hydrate), debromohymenialdisine and analogs, and VRX0466617. Other suitable Chk2 inhibitors are those described in Velic, D. et al., Biomolecules, 2015, 5, 3204-3259; Garrett, M. D. et al., Trends Pharmacol. Sci. 2011, 32, 308-316, Lountos, G. T. et al., J. Struct. Biol. 2011, 176, 292-301; Pommier, Y. et al., Clin. Cancer Res. 2006, 12, 2657-2661; and Hirao, A. et al., Science 2000, 287, 1824-1827, each of which is incorporated herein by reference in its entirety.

In certain embodiments, the Chk1/2 inhibitor that can be used in the methods of the present invention is LY2606368, SCH90076, LY2603618 or AZD7762.

Mouse double minute 2 homolog (Mdm2), also known as E3 ubiquitin-protein ligase Mdm2, is a protein that in humans is encoded by the MDM2 gene. Mdm2 is an important negative regulator of the p53 tumor suppressor. Mdm2 protein functions both as an E3 ubiquitin ligase that recognizes the N-terminal trans-activation domain (TAD) of the p53 tumor suppressor and as an inhibitor of p53 transcriptional activation.

“Mdm2 inhibitor” refers to substances that inhibit the activity of Mdm2.

Suitable Mdm2 inhibitors that can be used in the present invention include, but are not limited to, nutlin-3, RG7112, R05503781, SAR405838, DS-3032b, CGM-097, HDM201, MK4828, AMG232, RG7388, nutlin-3, JNJ26854165, and ALRN-6924 (Buress, A. et al., Front Oncol. 2016, 6: 7, incorporated herein by reference in its entirety). In certain embodiments, the Mdm2 inhibitor is nutlin-3.

### Therapeutic Applications

The present invention provides methods for treating patients with cancer, in particular a hematologic cancer, such as AML, by administering a combination of a CD33-targeted ADC and cytarabine. In other embodiments, the present invention also provides methods for treating patients with cancer, in particular, hematologic cancer, by administering a combination of a CD33-targeted ADC and a Chk1/2 inhibitor. In yet other embodiments, the present invention also provides methods for treating patients with cancer, in particular, hematologic cancer, by administering a combination of a CD33-targeted ADC and a Mdm2 inhibitor. As used herein, a “hematologic cancer” is a cancer that begins in blood-forming tissue, such as the bone marrow, or in the cells of the immune system. Examples of hematologic cancer are leukemia, lymphoma and multiple myeloma.

Cancers which can be treated using the disclosed methods include leukemia, lymphoma and myeloma. The cancer can be chemotherapy sensitive; alternatively, the cancer can be chemotherapy resistant. More specifically, cancers which can be treated using the disclosed methods include acute lymphoblastic leukemia (ALL), acute myeloid leukemia (AML), chronic lymphocytic leukemia (CLL), chronic myelogenous leukemia (CML), acute pro-myelocytic leukemia (APL), myelodysplastic syndromes (MDS), acute monocytic leukemia (AMOL), hairy cell leukemia (HCL), T-cell prolymphocytic leukemia (T-PLL), large granular lymphocytic leukemia, adult T-cell leukemia, small lymphocytic lymphoma (SLL), Hodgkin's lymphomas (Nodular sclerosis, Mixed cellularity, Lymphocyte-rich, Lymphocyte depleted or not depleted, and Nodular lymphocyte-predominant Hodgkin lymphoma), non-Hodgkin's lymphomas (all subtypes), chronic lymphocytic leukemia/Small lymphocytic lymphoma, B-cell prolymphocytic leukemia, lymphoplasmacytic lymphoma (such as Waldenström macroglobulinemia), splenic marginal zone lymphoma, plasma cell neoplasms (plasma cell myeloma, plasmacytoma, monoclonal immunoglobulin deposition diseases, heavy chain diseases), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, Aggressive NK cell leukemia, Adult T cell leukemia/lymphoma, extranodal NK/T cell lymphoma (nasal type), enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma (unspecified), anaplastic large cell lymphoma), and multiple myeloma (plasma cell myeloma Kahler's disease).

In another embodiment, the cancer is selected from acute myeloid leukemia (AML), chronic myeloid leukemia (CML), acute lymphoblastic leukemia (ALL), B-cell lineage acute lymphoblastic leukemia (B-ALL), T-cell lineage acute lymphoblastic leukemia (T-ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, eosinophilic leukemia, B myelomonocytic leukemia and Hodgkin's leukemia (HL). In another embodiment, the cancer is acute myeloid leukemia (AML). In certain embodiments, the subject is a fit AML subject; while in other embodiments, the subject is an unfit AML subject. In yet another embodiment, the acute myeloid leukemia is refractory or relapsed acute myeloid leukemia. In other embodiments, the invention provides treatment of patients with multi-drug resistant AML. P-glycoprotein (PGP), also known as MDR1, is an ATP-dependent drug efflux pump of 170 kD. It is a member of the ABC superfamily and is abundantly expressed in multidrug resistance (MDR) cells and produced by the ABCB1 gene. AML cells expressing PGP are, at least to some degree, resistant to treatment with conventional chemotherapeutics. Thus, the invention also provides methods for treating PGP-expressing AML.

The invention also provides methods of treating a hematologic cancer having at least one negative prognostic factor, e.g., overexpression of P-glycoprotein, overexpression of EVIL a p53 alteration, DNMT3A mutation, FLT3 internal tandem duplication, and/or complex karyotype. In other embodiments, the invention also provides methods of treating a hematologic cancer having decreased expression in BRCA1, BRCA2, or PALB2 or mutations in BRCA1, BRCA2, or PALB2. Also within the scope of the invention is the selection of patients having at least one negative prognostic factor and/or decreased expression or mutations in BRCA1, BRCA2, or PALB2 prior to administration of the combination of a CD33 targeted ADC described herein and cytarabine, the combination of a CD33 targeted ADC described herein and a Chk1/2 inhibitor, or the combination of a CD33 targeted ADC described herein and a Mdm2 inhibitor.

In particular embodiments, the CD33-targeted ADC is administered to a subject in a pharmaceutically acceptable dosage form. ADCs may be administered intravenously as a bolus or by continuous infusion over a period of time, by intramuscular, subcutaneous, intraarticular, intrasynovial, intrathecal, oral, topical, or inhalation routes. Pharmaceutical compositions containing ADCs are administered by intratumoral, peritumoral, intralesional, or perilesional routes, to exert local as well as systemic therapeutic effects.

A pharmaceutically acceptable dosage form will generally include a pharmaceutically acceptable agent such as a carrier, diluent, and excipient. These agents are well known and the most appropriate agent can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose.

In the disclosed methods, the ADC and cytarabine, the ADC and a Chk1/2 inhibitor, the ADC and a Mdm2 inhibitor are administered in combination. A combination therapy is meant to encompass administration of the two or more therapeutic agents to a single subject, and are intended to include treatment regimens in which the agents are administered by the same or different route of administration or at the same or different times. These terms encompass administration of two or more agents to the subject so that both agents and/or their metabolites are present in the subject at the same time. They include simultaneous administration in separate compositions, simultaneous administration in the same composition and administration at different times in separate compositions.

In certain embodiments, cytarabine and the ADC can be administered to the subject concurrently.

In certain embodiments, cytarabine is administered to the subject before the administration of the ADC (e.g., IMGN779). In certain embodiments, cytarabine and the ADC (e.g., IMGN779) are administered to the subject on the same day. In other embodiments, cytarabine is administered prior to (e.g., 1 hour, 2 hours, 5 hours, 8 hours or 12 hours prior to) the administration of the ADC. In other embodiments, cytarabine is administered to the subject 1 day, 2 days, 3 days, 4 days, 5 days, 6 days, or 7 days before the administration of the ADC (e.g., IMGN779). In certain embodiments, cytarabine is administered to the subject after the administration of the ADC (e.g., IMGN779), e.g., 1 day, 2 days, 3 days, 4 days, 5 days, 6 days, or 7 days after the administration of the ADC (e.g., IMGN779).

In certain embodiments, the combination of cytarabine and the ADC (e.g., IMGN779) can be used as a front line therapy for treating AML in a fit AML subject. In certain embodiments, the combination of cytarabine and the ADC (e.g., IMGN779) can be used as a second line therapy for treating AML in a fit AML subject. In certain embodiments, the combination of cytarabine and the ADC (e.g., IMGN779) can be used for treating relapsed or refractory AML in a fit AML subjects. In certain embodiments, the combination of cytarabine and the ADC (e.g., IMGN779) can be used as a second line therapy for treating relapsed or refractory AML in a fit AML subjects.

In certain embodiments, the combination of cytarabine and the ADC (e.g., IMGN779) can be used as a front line therapy for treating AML in an unfit AML subject. In certain embodiments, the combination of cytarabine and the ADC (e.g., IMGN779) can be used as a second line therapy for treating AML in an unfit AML subject. In certain embodiments, the combination of cytarabine and the ADC (e.g., IMGN779) can be used for treating relapsed or refractory AML in an unfit AML subjects. In certain embodiments, the combination of cytarabine and the ADC (e.g., IMGN779) can be used as a second line therapy for treating relapsed or refractory AML in an unfit AML subjects.

The ADCs used in the disclosed methods and pharmaceutical compositions can be supplied as a solution or a lyophilized powder that are tested for sterility and for endotoxin levels. Suitable pharmaceutically acceptable carriers, diluents, and excipients are well known and can be determined by those of ordinary skill in the art as the clinical situation warrants.

Examples of suitable carriers, diluents and/or excipients include: (1) Dulbecco's phosphate buffered saline, pH about 7.4, containing or not containing about 1 mg/ml to 25 mg/ml human serum albumin, (2) 0.9% saline (0.9% w/v NaCl), and (3) 5% (w/v) dextrose; and may also contain an antioxidant such as tryptamine and a stabilizing agent such as Tween 20.

Pharmaceutical compositions are disclosed that include an ADC, cytarabine, and typically at least one additional substance, such as a pharmaceutically acceptable carrier or diluent. In some embodiments, pharmaceutical compositions are also disclosed that include an ADC, a Chk1/2 inhibitor, and typically at least one additional substance, such as a pharmaceutically acceptable carrier or diluent. In other embodiments, pharmaceutical compositions are disclosed that include an ADC, a MDM2 inhibitor, and typically at least one additional substance, such as a pharmaceutically acceptable carrier or diluent. The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1. In Vivo Efficacy of Combination of IMGN779 and Cytarabine in EOL-1 Subcutaneous Model

### Data Collection and Analysis for all Subcutaneous Xenograft Models:

The mice were weighed twice a week and were monitored for clinical signs throughout the duration of the study. Animals were euthanized when hind leg paralysis was present, body weight decreased by >20% of pre-treatment weight, tumor ulceration occurred, or when any signs of distress were visible. Tumor volumes were measured one to two times weekly in three dimensions using a caliper. The tumor volume was expressed in mm³ using the formula V=Length×Width×Height×½ (Tomayko and Reynolds, Cancer Chemother. Pharmacol. 24: 148-54 (1989)). Activity was assessed as described in Bissery et al., Cancer Res. 51: 4845-52 (1991). Tumor Growth Inhibition (T/C Value) was also assessed using the following formula: T/C (%)=(Median tumor volume of the treated/Median tumor volume of the control)×100%. Tumor volume was determined simultaneously for the treated (T) and the vehicle control (C) groups when tumor volume of the vehicle control reached a predetermined size (Bissery et al., Cancer Res. 51: 4845-52 (1991). The daily median tumor volume of each treated group was determined, including tumor-free mice (0 mm³). According to NCI standards, a T/C≤42% is the minimum level of anti-tumor activity. A T/C<10% is considered a high anti-tumor activity level.

To test the efficacy of IMGN779 in combination with cytarabine for the ability to decrease tumor burden in vivo, a subcutaneous tumor model was used as described in the protocol below.

Female athymic nude mice were each inoculated with 1×10⁷ EOL-1 cells, a human AML cell line, in 100 μl serum free medium subcutaneously in the right flank. On day 9, which is 24 h prior to conjugate administration for groups receiving conjugate treatment (either alone or in combination with cytarabine), all the mice in these treatment groups were injected intraperitoneally with 400 mg/kg of non-targeted chKTI antibody to block Fc receptors on the EOL-1 AML cells, preventing non-specific up-take of conjugate. Additionally, on day 14 post-cell inoculation, all mice receiving conjugate received a second injection of 100 mg/kg of chKTI antibody again to block Fc receptors. On day 10 post-EOL-1 inoculation, mice were randomized into the study groups based on tumor volume.

On day 10 post-EOL-1 inoculation, the mice received a single intravenous injection, in the lateral tail vein, of vehicle, 5 μg/kg (by DGN462; 0.253 mg/kg by huCD33 Ab) IMGN779. Cytarabine administration was also initiated on day 10; and the mice receiving cytarabine were given a single intraperitoneal injection of 75 mg/kg cytarabine on each day of days 10, 11, 12, 13 and 14 post-cell implantation. In the combination group, the mice received administrations of both IMGN779 and cytarabine as outlined above. The results are represented in Table 3 (below) and in FIG. 1.

A single dose of 5 μg/kg IMGN779 was highly active in this study, resulting in a T/C value of 5% and 2/6 long-term complete regressions (CRs) at the end of the study (day 90). In contrast, a regimen of 75 mg/kg of cytarabine, once-a-day (qd) for five days (×5), was inactive, resulting in a T/C of 95% and 0/6 CRs on day 90. The combination of a single dose of IMGN779 and the QD×5 regimen of cytarabine was highly active, resulting in a T/C of 0% and 4/6 CRs on day 90. Note that the combination regimen resulted in two additional long-term CRs out of six mice (4/6 CRs) when compared to treatment with IMGN779 alone (2/6 CRs), demonstrating the additional benefit derived from treatment with the combination regimen.

TABLE 3

| | | | | | Tumor | |
|---|---|---|---|---|---|---|
| | | % T/C | | | Free | |
| | | (Day | | | Survivors | |
| Group/Treatment | Treatment Days | 19) | PR | CR | (Day 90) | Result |
| A) Vehicle | As below | — | 0/6 | 0/6 | 0/6 | — |
| B) IMGN779, 5 μg/kg | Day 10 | 5 | 4/6 | 2/6 | 2/6 | Highly |
| | | | | | | Active |
| C) Cytarabine, 75 mg/kg | Days | 95 | 0/6 | 0/6 | 0/6 | Inactive |
| | 10, 11, 12, 13, 14 | | | | | |
| D) Combination of | IMGN779: | 0 | 6/6 | 6/6 | 4/6 | Highly |
| IMGN779 (5 μg/kg) | Day 10; | | | | | Active |
| and Cytarabine (75 mg/kg) | Cytarabine: | | | | | |
| | Days | | | | | |
| | 10, 11, 12, 13, 14 | | | | | |

### Example 2. In Vivo Efficacy of Combination of IMGN779 (QW×3) and Cytarabine in MV4-11 Disseminated Model

### Data Collection and Analysis for all Disseminated Xenograft Models:

The mice were weighed twice a week and were monitored for clinical signs throughout the duration of the study. The measured end-point was survival. Animals were euthanized when hind leg paralysis was present, body weight decreased by >20% of pre-treatment weight, a visible tumor appeared, or any signs of distress were visible. Spontaneous deaths were recorded when they were discovered. For disseminated models, Tumor Growth Delay is calculated as T−C, where T is the median survival time (in days) of a treated group and C is the median survival time (in days) of the vehicle control group. The Percent Increased Life Span (% ILS) for disseminated models is calculated using the following formula: % ILS=(T−C)/C×100%. Anti-tumor activity was evaluated as per NCI standards for disseminated models: ILS≥25% is minimum active, ILS>40% is active, and ILS≥50% is highly active.

To test the efficacy of IMGN779, cytarabine or the combination of these two agents for the ability to decrease tumor burden in vivo, a disseminated tumor model was used as described in the protocol below.

Female NOD SCID mice were pre-treated with 150 mg/kg cyclophosphamide to partially ablate bone marrow in order to improve the engraftment of MV4-11 cells. The cyclophosphamide (Sigma, C0768, Lot # MKBX1822V) was dissolved in 0.9% NaCl and was administered intraperitoneally to the mice on day −3 and day −2 prior to MV4-11 cell inoculation.

Following cyclophosphamide treatment as described above, the mice were each injected intravenously in the lateral tail vein with 3×10⁶ MV4-11 cells, a human AML cell line, in 100 μl of serum-free medium on day 0 in the study. On day 20 post-MV4-11 inoculation, mice were randomized into the study groups based on body weight. At 24 h prior to each conjugate administration for all groups receiving conjugate treatment (either alone or in combination with cytarabine), all the mice in these treatment groups except for those in the Group L were injected intraperitoneally with 150 mg/kg of non-targeted chKTI antibody to block Fc receptors on the MV4-11 AML cells, preventing non-specific up-take of conjugate.

Mice received IMGN779, at a dose of 1 μg/kg by DGN462 (0.0534 mg/kg by anti-huCD33 antibody), once a week (QW) for three doses (×3) according to four different dosing schedules, either alone or in combination with cytarabine. The four different IMGN779 dosing schedules were: i) days 21, 28 and 35 (referred to as “Day 21”); ii) days 24, 31 and 38 (referred to as “Day 24”); iii) days 28, 35 and 42 (referred to as “Day 28”); and iv) days 31, 38 and 45 (referred to as “Day 31”); where the noted start day of IMGN779 treatment was moved progressively further away from day 0 (the day of MV4-11 inoculation) in the study time line. Cytarabine was given once a day (QD) at a dose of 75 mg/kg for five consecutive days (×5) and always administered on the same fixed set of days in the study time line: days 24, 25, 26, 27 and 28.

An additional IMGN779 treatment group was included in this study (Group L), where FcR blocking was not done, and where the mice were dosed with 1 μg/kg by DGN462 of IMGN779, QW×3, on a day 21, 28 and 35 dosing schedule (“Day 21”). This was done in order to determine whether or not a detectable increased level of anti-tumor activity resulted from FcR-mediated non-specific (e.g. not CD33-targeted) uptake of the low doses of IMGN779 used in this study. Lastly, Group K received QW×3 dosing of 1 μg/kg by DGN462 (0.0556 mg/kg by antibody) of a non-targeted control conjugate, Ab-DGN462, on a day 21, 28 and 35 dosing schedule (“Day 21”, with FcR blocking). Group K was included in order to determine the level of non-CD33 targeted anti-tumor activity inherent within the corresponding monotherapy regimen of 1 μg/kg (by DGN462) of IMGN779, where IMGN779 was also administered on days 21, 28 and 35 (“Day 21”, Group C).

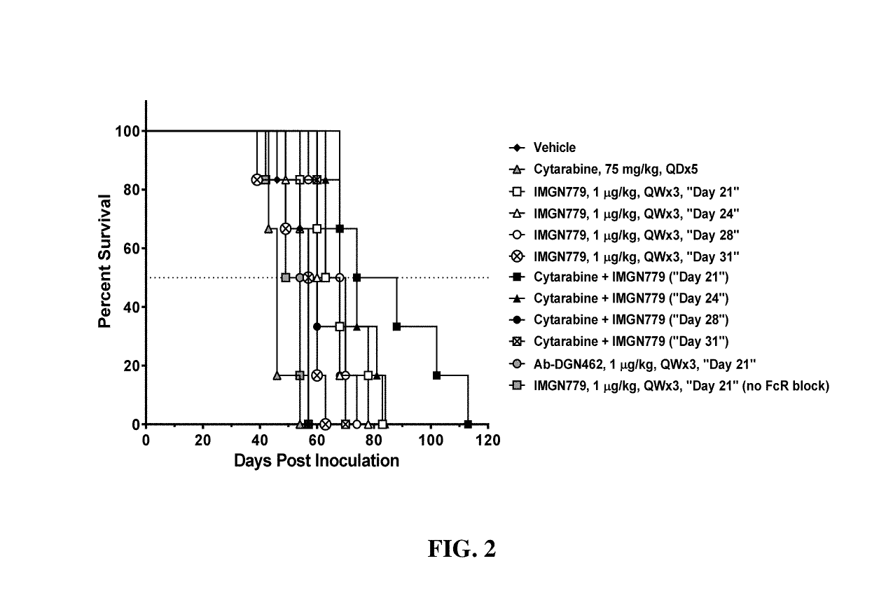

Mice treated with a combination regimen of IMGN779, according to each one of the IMGN779 dose schedules outlined above, and 75 mg/kg cytarabine (QD×5; days 24, 25, 26, 27, 28) received treatments on the same days as their corresponding monotherapy comparator groups outlined above. The results are represented in Table 4 (below) and in FIG. 2.

Cytarabine administered at 75 mg/kg, QD×5, was inactive in this study, resulting in a tumor growth delay (T−C value) of 0 days and a 0% ILS (Increased Life Span) compared to vehicle treatment. Each of the four different treatment schedules (defined by the first day IMGN779 dosing began; outlined above) of single agent IMGN779, each schedule dosed at QW×3, was inactive. The four inactive IMGN779 single agent treatment regimens resulted in the following T−C values and % ILS: i) the “Day 21” schedule: a T−C of 8.5 days and a 15% ILS; ii) the “Day 24” schedule: a T−C of 7 days and a 12.3% ILS; iii) the “Day 28” schedule: a T−C of 12 days and a 21% ILS, and iv) the “Day 31” schedule: a T−C of 1.5 days and a 2.6% ILS.

The four cytarabine plus IMGN779 combination therapy regimens, where the IMGN779 treatment was delivered over four different schedules outlined above, resulted in the following anti-tumor activity: i) a T−C value of 24 days and a 42% ILS (active) when the IMGN779 regimen began on Day 21, ii) a T−C value of 17 days and a 29.8% ILS (minimally active) when the IMGN779 regimen began on Day 24, iii) a T−C value of 3 days and a 5.3% ILS (inactive) when the IMGN779 regimen began on Day 28, and iv) a T−C value of 8.5 days and a 15% ILS (inactive) when the IMGN779 regimen began on Day 31.

Single agent treatment with a QW×3 regimen of 1 μg/kg (by DGN462; 0.0556 mg/kg by antibody) of the non-targeted Ab-DGN462 control conjugate (Group K), beginning treatment on Day 21, resulted in a T−C value of 0 days and in a 0% ILS, which was inactive, which is a similar outcome to the corresponding dose schedule of IMGN779 single agent (Group C). Single agent treatment with a QW×3 regimen of IMGN779, beginning treatment on Day 21 but without FcR blocking (Group L), was also inactive, resulting in a T−C value of 0 days and a 0% ILS, indicating no increased IMGN779 up-take without FcR blocking.

TABLE 4

| | | | Tumor | | |
|---|---|---|---|---|---|
| | | Median | Growth | % | |
| | | Survival | Delay | Increased | |
| | Treatment | Time | (T-C, | Life Span | |
| Group/Treatment | Days | (Days) | Days) | (ILS) | Result |
| A) Vehicle | As below | 57 | — | — | — |
| B) Cytarabine, 75 mg/kg | Days | 46 | 0 | 0 | Inactive |
| | 24, 25, 26, 27, 28 | | | | |
| C) IMGN779, 1 μg/kg | Days 21, 28, 35 | 65.5 | 8.5 | 15 | Inactive |
| (“Day 21”) | | | | | |
| D) IMGN779, 1 μg/kg | Days 24, 31, 38 | 64 | 7 | 12.3 | Inactive |
| (“Day 24”) | | | | | |
| E) IMGN779, 1 μg/kg | Days 28, 35, 42 | 69 | 12 | 21 | Inactive |
| (“Day 28”) | | | | | |
| F) IMGN779, 1 μg/kg | Days 31, 38, 45 | 58.5 | 1.5 | 2.6 | Inactive |
| (“Day 31”) | | | | | |
| G) #1 Combination of | Cytarabine as | 81 | 24 | 42 | Active |
| Cytarabine (75 mg/kg) and | above; | | | | |
| IMGN779 (1 μg/kg; “Day | IMGN779 | | | | |
| 21”) | days 21, 28, 35 | | | | |
| H) #2 Combination of | Cytarabine as | 74 | 17 | 29.8 | Minimally |
| Cytarabine (75 mg/kg) and | above; | | | | Active |
| IMGN779 (1 μg/kg; “Day | IMGN779 | | | | |
| 24”) | days 24, 31, 38 | | | | |
| I) #3 Combination of | Cytarabine as | 60 | 3 | 5.3 | Inactive |
| Cytarabine (75 mg/kg) and | above; | | | | |
| IMGN779 (1 μg/kg; “Day | IMGN779 | | | | |
| 28”) | days 28, 35, 42 | | | | |
| J) #4 Combination of | Cytarabine as | 65.5 | 8.5 | 15 | Inactive |
| Cytarabine (75 mg/kg) and | above; | | | | |
| IMGN779 (1 μg/kg; “Day | IMGN779 | | | | |
| 31”) | days 31, 38, 45 | | | | |
| K) Ab-DGN462, 1 μg/kg | Days 21, 28, 35 | 55.5 | 0 | 0 | Inactive |
| L) IMGN779, 1 μg/kg; | Days 21, 28, 35 | 51.5 | 0 | 0 | Inactive |
| “Day 21”, with no FcR | | | | | |
| blocking | | | | | |

### Example 3. In Vivo Efficacy of Combination of IMGN779 (QW×3) and Cytarabine in Molm-13 Disseminated Model

To test the efficacy of IMGN779, cytarabine or the combination of these two agents for the ability to decrease tumor burden in vivo, a disseminated tumor model was used as described in the protocol below.

Female NOD SCID mice were pre-treated with 150 mg/kg cyclophosphamide to partially ablate bone marrow in order to improve the engraftment of Molm-13 cells. The cyclophosphamide (Sigma, C0768, Lot # MKBX1822V) was dissolved in 0.9% NaCl and was administered intraperitoneally to the mice on day −2 prior to Molm-13 cell inoculation on day 0.

Following cyclophosphamide treatment as described above, the mice were each injected intravenously in the lateral tail vein with 2×10⁵ Molm-13 cells, a human AML cell line, in 100 μl of serum-free medium on day 0 in the study. On day 6 post-Molm-13 inoculation, mice were randomized into the study groups based on body weight. At 24 h prior to each conjugate administration, all groups receiving conjugate treatment (either alone or in combination with cytarabine), except for Groups I and J (which remained unblocked), were injected intraperitoneally with 150 mg/kg of non-targeted chKTI antibody to block Fc receptors on the Molm-13 AML cells, preventing non-specific up-take of conjugate.

Mice received IMGN779, at a dose of 0.2 μg/kg by DGN462 (0.0107 mg/kg by anti-huCD33 antibody), once a week (QW) for three doses (×3) according to three different dosing schedules, either alone or in combination with cytarabine. The three different IMGN779 dosing schedules were: i) days 7, 14 and 21 (referred to as “Day 7”); ii) days 11, 18 and 25 (referred to as “Day 11”); iii) days 14, 21 and 35 (referred to as “Day 14”); where the noted start day of IMGN779 treatment was moved progressively further away from day 0 (the day of Molm-13 inoculation) in the study time line. Cytarabine, in contrast, was given once a day (QD) at a dose of 75 mg/kg for five consecutive days (×5) and always on a fixed set of days in the study time line: Days 7, 8, 9, 10 and 11.

An IMGN779 treatment group was included in this study (Group I), where FcR blocking was not done, and where the mice were dosed with 0.2 μg/kg by DGN462 of IMGN779, QW×3, on a day 7, 14 and 21 dosing schedule (“Day 7”). The purpose of Group I was to determine whether or not a detectable level of increased anti-tumor activity resulted from the possible additional contribution of FcR-mediated non-specific (e.g. not CD33-targeted) uptake of the low doses of IMGN779 used in this study by comparing Group I (unblocked) to Group C, which had the same regimen and schedule of IMGN779 but which received FcR blocking. Lastly, Group J received QW×3 dosing of 0.2 μg/kg by DGN462 (0.0111 mg/kg by antibody) of a non-targeted control conjugate, Ab-DGN462, on a day 7, 14 and 21 dosing schedule (“Day 7”; with FcR blocking) in order to determine the level of non-CD33 targeted anti-tumor activity inherent within the corresponding monotherapy regimen of 0.2 μg/kg IMGN779, where IMGN779 was administered also on days 7, 14 and 21, but with FcR blocking.

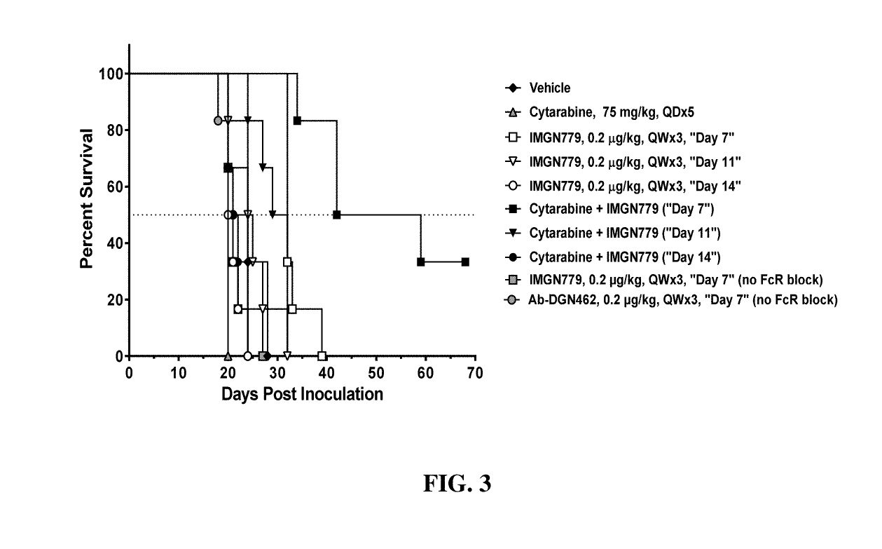

Mice treated with a combination regimen of IMGN779, according to each one of the IMGN779 dose schedules outlined above, and 75 mg/kg cytarabine (QD×5; days 7, 8, 9, 10 and 11) received treatments on the same days as their corresponding monotherapy comparator groups outlined above. The results are represented in Table 5 (below) and in FIG. 3.

Cytarabine administered at 75 mg/kg, QD×5, was inactive in this study, resulting in a tumor growth delay (T−C value) of 0 days and a 0% ILS (Increased Life Span) compared to vehicle treatment. The three IMGN779 single agent treatment regimens resulted in the following T−C values, % ILS and activity results: i) the “Day 7” schedule: a T−C of 8 days and a 33% ILS, which was minimally active; ii) the “Day 11” schedule: a T−C of 0.5 days and a 2% ILS, which was inactive; iii) the “Day 14” schedule: a T−C of 0 days and a 0% ILS, which was inactive.

The three Cytarabine plus IMGN779 combination therapy regimens, where the IMGN779 treatment was delivered over three different schedules outlined above, resulted in the following anti-tumor activity: i) a T−C value of 26.5 days and a 110% ILS (highly active) when the IMGN779 regimen began on Day 7, ii) a T−C value of 6.5 days and a 27% ILS (minimally active) when the IMGN779 regimen began on Day 11, iii) a T−C value of 0 days and a 0% ILS (inactive) when the IMGN779 regimen began on Day 14.

Single agent treatment with a QW×3 regimen of IMGN779, beginning treatment on Day 7, but without FcR blocking (Group I), was inactive, resulting in a T−C value of 0 days and a 0% ILS, which was a similar outcome to that of the corresponding FcR blocked dosing schedule of IMGN779 single agent (Group C). Single agent treatment with a QW×3 regimen of 0.2 μg/kg by DGN462 (0.0111 mg/kg by antibody) of the non-targeted Ab-DGN462 control conjugate (Group J), beginning treatment on Day 7 and with FcR blocking, resulted in a T−C value of 0 days and in a 0% ILS, which was inactive, which is a similar outcome to the corresponding FcR blocked dosing schedule of IMGN779 single agent (Group C).

TABLE 5

| | | | Tumor | % | |
|---|---|---|---|---|---|
| | | Median | Growth | Increased | |
| | | Survival | Delay | Life | |
| | | Time | (T-C, | Span | |
| Group/Treatment | Treatment Days | (Days) | Days) | (ILS) | Result |
| A) Vehicle | As below | 24 | — | — | — |
| B) Cytarabine, 75 mg/kg | Days 7, 8, 9, 10, 11 | 20 | 0 | 0 | Inactive |
| C) IMGN779, 0.2 μg/kg | Days 7, 14, 21 | 32 | 8 | 33 | Minimally |
| (“Day 7”) | | | | | Active |
| D) IMGN779, 0.2 μg/kg | Days 11, 18, 25 | 24.5 | 0.5 | 2 | Inactive |
| (“Day 11”) | | | | | |
| E) IMGN779, 0.2 μg/kg | Days 14, 21, 28 | 20.5 | 0 | 0 | Inactive |
| (“Day 14”) | | | | | |
| F) #1 Combination of | Cytarabine: Days | 50.5 | 26.5 | 110 | Highly |
| Cytarabine (75 mg/kg) and | 7, 8, 9, 10, 11; and | | | | Active |
| IMGN779 (0.2 μg/kg; | IMGN779: 7, 14, 21 | | | | |
| “Day 7”) | | | | | |
| G) #2 Combination of | Cytarabine: Days | 30.5 | 6.5 | 27 | Minimally |
| Cytarabine (75 mg/kg) and | 7, 8, 9, 10, 11; and | | | | Active |
| IMGN779 (0.2 μg/kg; | IMGN779 Days | | | | |
| “Day 11”) | 11, 18, 25 | | | | |
| H) #3 Combination of | Cytarabine: Days | 21.5 | 0 | 0 | Inactive |
| Cytarabine (75 mg/kg) and | 7, 8, 9, 10, 11; and | | | | |
| IMGN779 (0.2 μg/kg; | IMGN779 Days | | | | |
| “Day 14”) | 14, 21, 28 | | | | |
| I) IMGN779, 0.2 μg/kg; | Days 7, 14, 21 | 21 | 0 | 0 | Inactive |
| “Day 7”, with no FcR | | | | | |
| blocking | | | | | |
| J) Ab-DGN462, 0.2 μg/kg; | Days 7, 14, 21 | 22.5 | 0 | 0 | Inactive |
| “Day 7”, with no FcR | | | | | |
| blocking | | | | | |

### Example 4. In Vitro Evaluation of CD33 Expression Post-Treatment with Cytarabine

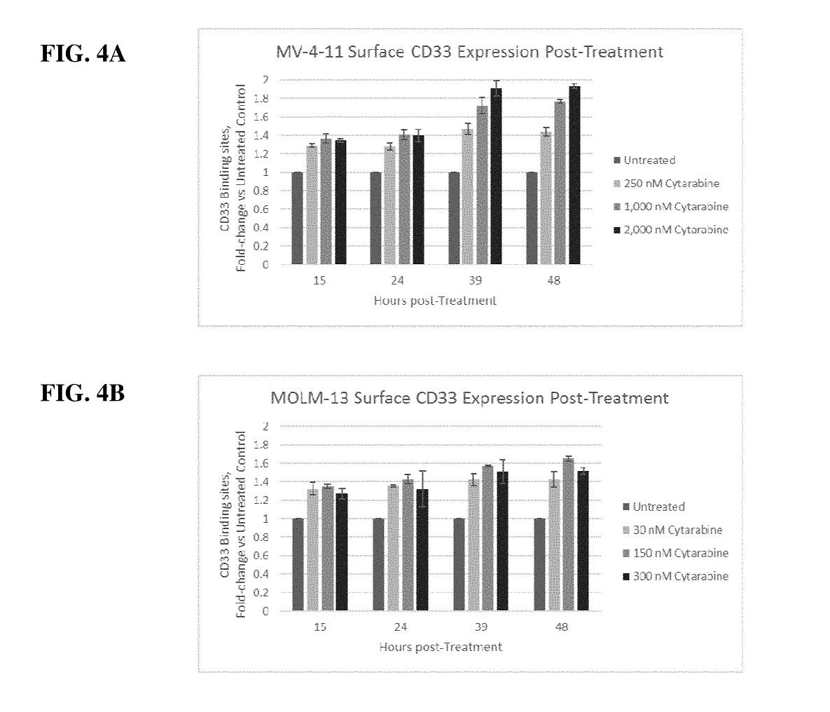

CD33 expression was assessed post-treatment with cytarabine. In these experiments, two AML cell lines with cell surface display of CD33 and cytarabine sensitivity were used: MV4-11 and MOLM-13. The potency-relative doses were chosen for each treatment, relative to IC50s generated from 96-hour cytotoxicity assays. For MV4-11, the concentration of IMGN779 used was 90 pM (IC50×50) and cytarabine concentration used was 2,500 nM (IC50×10). For MOLM-13, the concentration of IMGN779 used was 75 pM (IC50×150) and the concentration of cytarabine used was 400 nM (IC50×20). Time points at 15 hours, 24 hours, 39 hours, and 48 hours post-treatment with cytarabine were chosen for cell surface CD33 assessment. Cells were stained with, then washed of excess, PE-conjugated anti-CD33 mAb. Flow cytometric analyses of PE MFI were run on a FACSCanto II flow cytometer. PE-MFIs at each timepoint were normalized to PE-MFIs of untreated control samples and normalized PE-MFIs were considered to represent relative fold-changes in surface CD33 binding sites. The relative surface CD33 binding site values were graphed and results shown in FIGS. 4A-4B.

Exposure to cytarabine in vitro led to a dose- and time-dependent increase in CD33 transcription and cell surface expression in the two human AML cell lines tested, revealing a mechanism by which cytarabine may potentiate the CD33-dependent uptake of IMGN779 by AML cells.

### Example 5. In Vitro Apoptotic Response to IMGN779 and Cytarabine

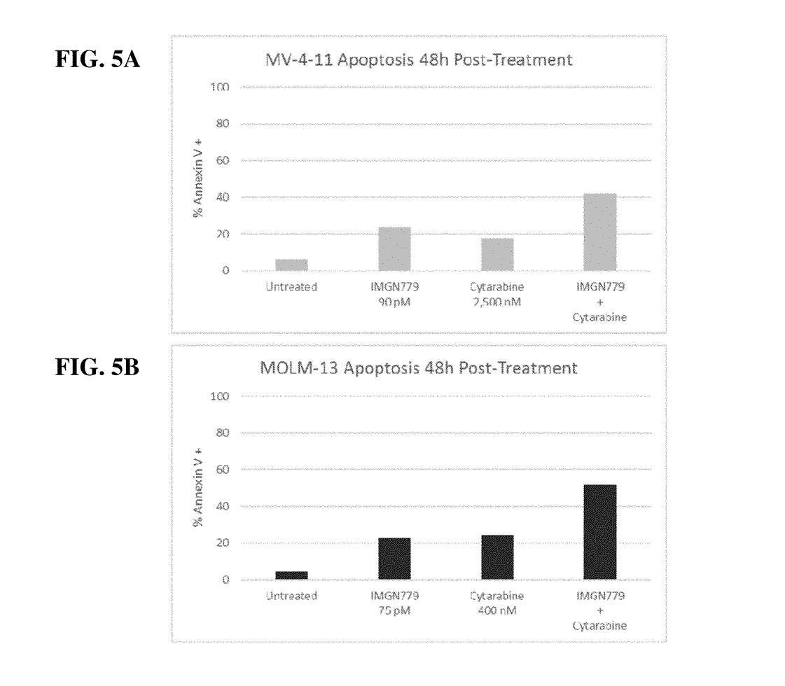

In vitro apoptotic response to the combination of IMGN779 and cytarabine was further assessed in MV4-11 and MOLM-13 cell lines. The potency-relative doses were chosen for each treatment as detailed above. One time point post-treatment at 48 hours was chosen for assessment of apoptosis. At 48 hours after treatment, cells were assessed for the percent of cells positive for the apoptosis marker, Annexin V. Cells were stained with AlexaFluor™ 488-conjugated Annexin V. Flow cytometric analyses of percent fluorescent cells were run on a FACSCanto II flow cytometer (FITC and APC channels). The analyses was carried out on FACSDiva software. Cells were determined by FSC/SSC gating. 20,000 “cells” events were collected per sample. Cells were plotted on a bivariate graph of “FITC” and “APC” fluorescence, divided into quadrants. The percentages from the two quadrants representing Annexin V-positive cells (“FITC+”) were summed and plotted in FIGS. 5A-5B.

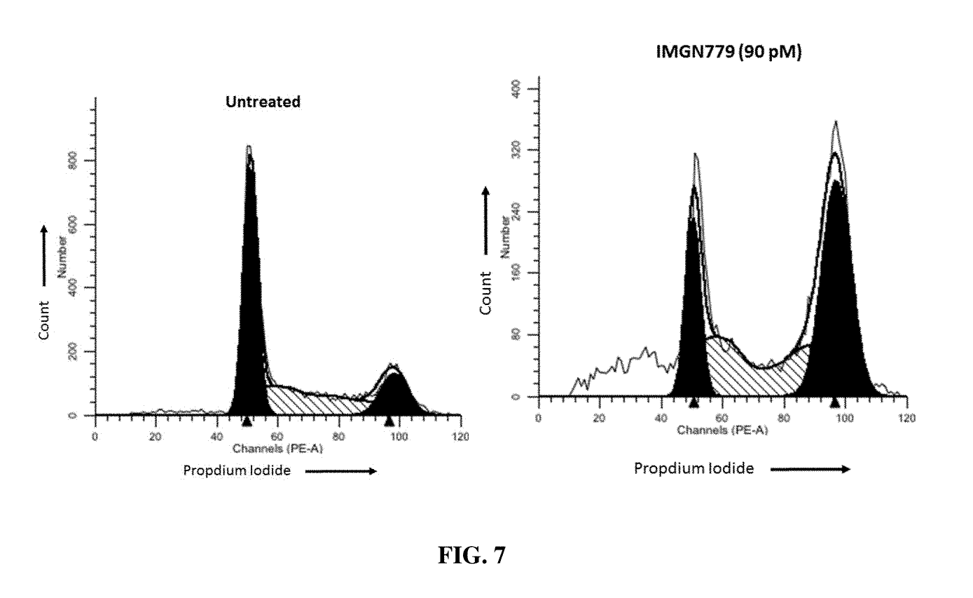

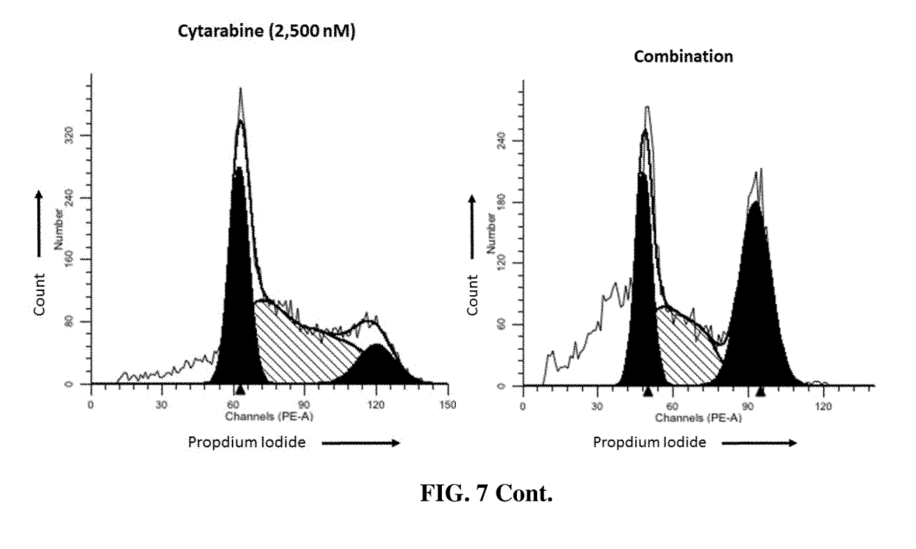

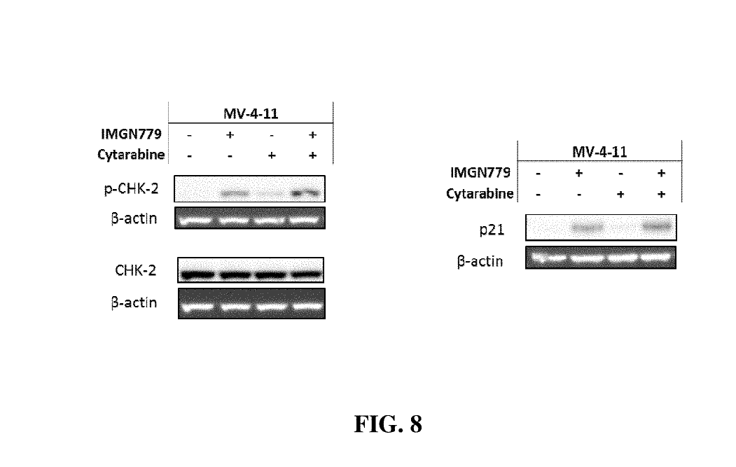

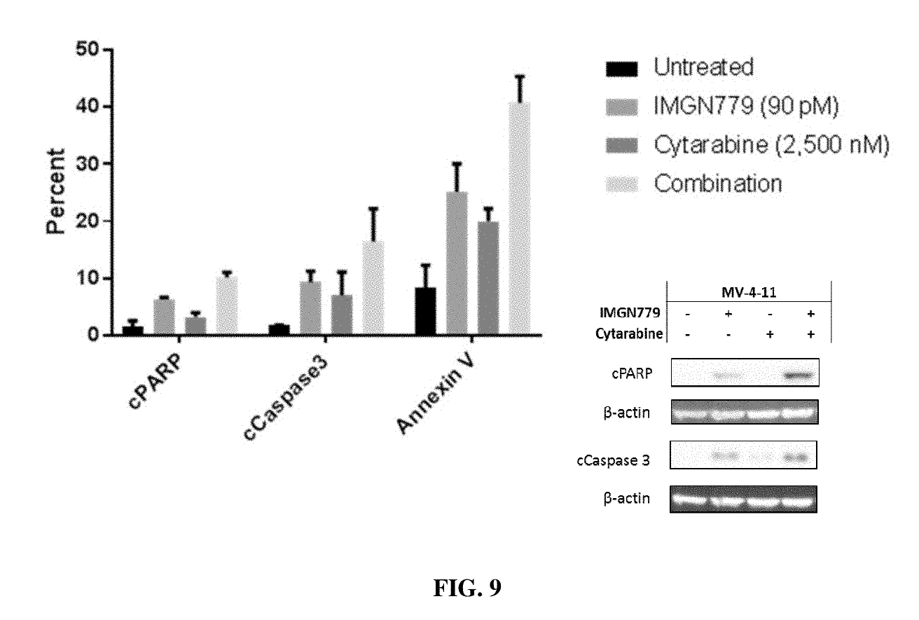

The in vitro combination of IMGN779 and cytarabine resulted in greater percentages of Annexin V-positive (apoptotic) cells than treatment with either single agent. The combination also enhanced levels of cell cycle arrest in both S and G2/M phases of the cell cycle and increased the Sub G0/G1 population due to the increased levels of apoptosis (FIG. 7), DNA damage response (as measured by p21, CHK2) (FIG. 8), and apoptosis as measured by Casp3/PARP1 cleavage (FIG. 9).

### Example 6. Mechanistic Importance of DNA Damage Response Pathways in IMGN779-Mediated Anti-Leukemia Activity

The mechanistic importance of DNA damage response pathways in IMGN779-mediated antileukemia activity was demonstrated further using the IGN free payload in an in vitro combination screen with a panel of 12 human AML cell lines (CMK, GDM-1, HEL92.1.7, HL-60, Kasumi-1, KG-1, MV4-11, NOMO-1, PL-21, SKM-1, TF-1α, and THP-1).

Briefly, each cell line was cultured and plated in optimal conditions based on growth characteristics in 384- or 1536-well tissue culture plates. Compounds were added to assay plates using a 6×6 or 6×8 dose matrix of DGN462-SMe and each combination agent. Concentration ranges were selected based on single agent activity of each molecule on a panel of cell lines. Cell viability was evaluated using an ATPLite assay (Perkin Elmer) following 72 hours of treatment. All data points were collected via automated processes, quality controlled, and analyzed using Horizon CombinatoRx proprietary Chalice software. Horizon utilizes growth inhibition (GI) as a measure of cell viability. GI is calculated by applying the following test and equation: If TbV0: 100 ð1−ð [symbol] T−V0=V0 [symbol] If T≥V0: 100 ð1−ð [symbol] T−V0=ð [symbol] [symbol] V−V0 where T is the signal for a test agent after 72 hours, V is the vehicle-treated control measure at 72 hours, and V0 is the vehicle control measure at time zero. A GI reading of 0% represents no growth inhibition where cells treated with compound (T) and (V) vehicle signals are the same. A GI of 100% represents complete growth inhibition or cytostatic conditions where cells treated by compound match the signal of V0. Compounds reaching GI 200% are considered cytotoxic and represent complete cell death. To quantitate the combination effects in excess of Loewe additivity, a scalar measure was used to characterize the strength of synergistic interaction termed the Synergy Score. Synergy Score ¼ log fX log fY Σ max 0ð [symbol] ; Idata ð [symbol] Idata−ILoewe The Synergy Score equation integrates the experimentally observed activity volume at each point in the matrix in excess of a model surface numerically derived from the activity of the component agents using the Loewe model for additivity. Additional terms in the Synergy Score equation are used to normalize for various dilution factors used for individual agents and to allow for comparison of synergy scores across the entire experiment.

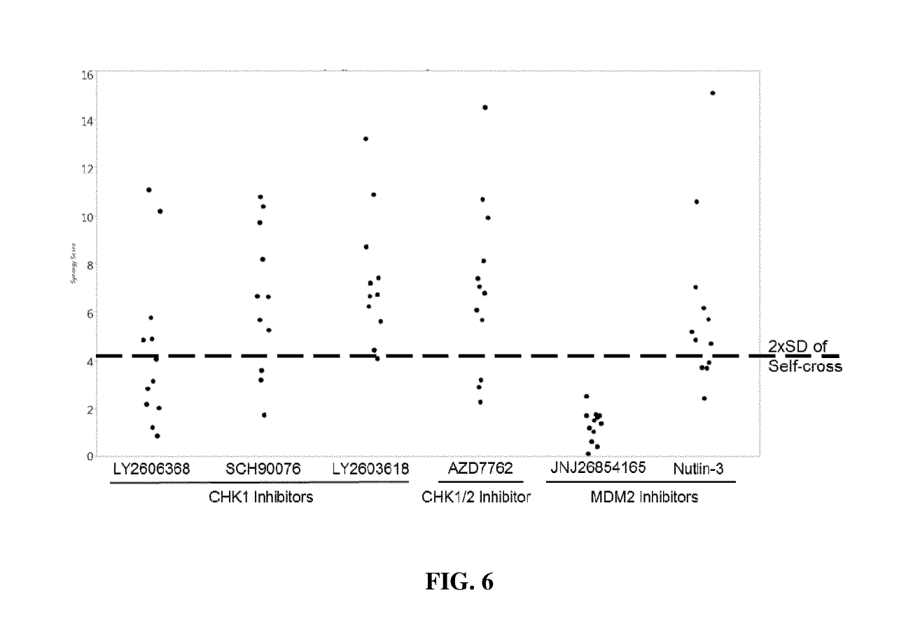

The results of this screen suggest strong synergy between IGN free payload and Chk1/2 inhibitors and Mdm2 inhibitors (FIG. 6), pointing to additional combination regimens for IMGN779.

1. A method of treating a cancer in a subject comprising administering to the subject an effective amount of cytarabine and an effective amount of an antibody-drug conjugate of Formula (I): 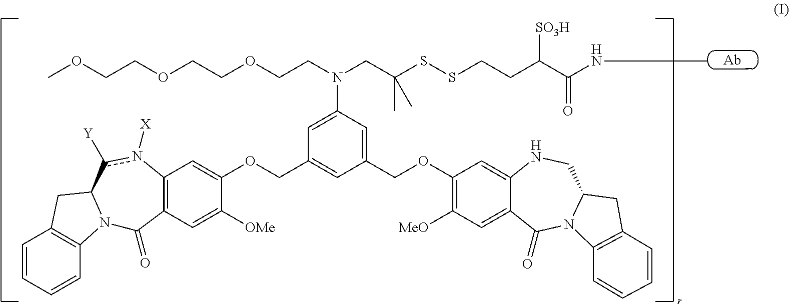
or a pharmaceutically acceptable salt thereof, wherein:
the double line [symbol] between N and C represents a single bond or a double bond, provided that when it is a double bond, X is absent and Y is hydrogen, when it is a single bond, X is hydrogen and Y is —SO₃H;
Ab is an anti-CD33 antibody or antigen-binding fragment thereof comprising a heavy chain variable region (V_{H}) complementary determining region (CDR)1 sequence of SEQ ID NO:1, a V_{H} CDR2 sequence of SEQ ID NO:2, and a V_{H} CDR3 sequence of SEQ ID NO:3, and a light chain variable region (V_{L}) CDR1 sequence of SEQ ID NO:4, a V_{L} CDR2 sequence of SEQ ID NO:5, and a V_{L} CDR3 sequence of SEQ ID NO:6; and
r is an integer from 1 to 10.

2. The method of claim 1, wherein the antibody-drug conjugate is represented by the following formula: 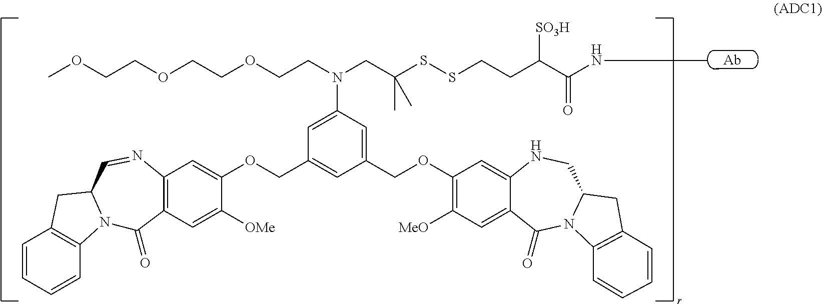
or a pharmaceutically acceptable salt thereof.

3. The method of claim 1, wherein the antibody-drug conjugate is represented by the following formula: 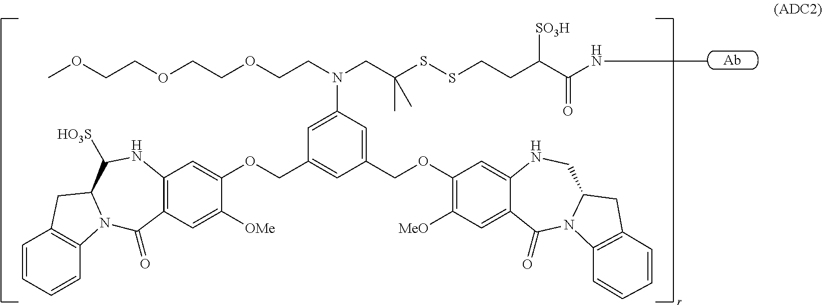
or a pharmaceutically acceptable salt thereof; and wherein the pharmaceutically acceptable salt is a sodium or potassium salt.

4. (canceled)

5. The method of claim 1, wherein the antibody-drug conjugate is represented by the following formula:

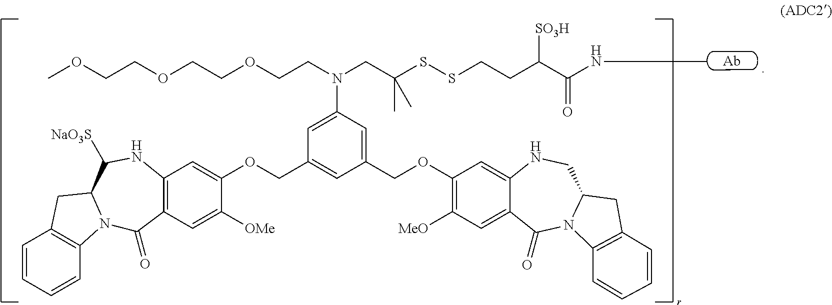

6. The method of claim 1, wherein the anti-CD33 antibody or antigen-binding fragment thereof comprises a heavy chain variable region comprising an amino acid sequence having at least 95% identity to the amino acid sequence of SEQ ID NO:7 or 9; and a light chain variable region comprising an amino acid sequence having at least 95% identity to the amino acid sequence of SEQ ID NO:8 or 10.

7. (canceled)

8. The method of claim 1, wherein the anti-CD33 antibody or antigen-binding fragment thereof comprises a heavy chain variable region comprising the sequence of SEQ ID NO:9 and a light chain variable region comprising the sequence of SEQ ID NO:10.

9. The method of claim 1, wherein Ab is an anti-CD33 antibody comprising a heavy chain having the amino acid sequence set forth in SEQ ID NO:11 and a light chain having the amino acid sequence set forth in SEQ ID NO:12.

10. The method of claim 1, wherein the antibody is a CDR-grafted or resurfaced antibody.

11. The method of claim 1, wherein the antibody-drug conjugate is represented by the following formula: 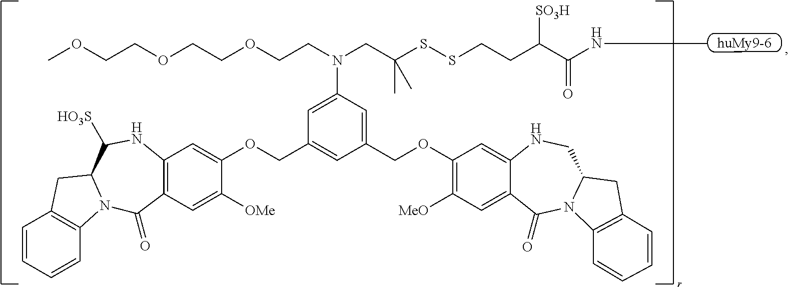
or a pharmaceutically acceptable salt thereof; and wherein the pharmaceutically acceptable salt is sodium salt.

12. (canceled)

13. The method of claim 1, wherein the cancer is selected from the group consisting of leukemia, lymphoma and myeloma.

14. The method of claim 13, wherein the cancer is selected from the group consisting of acute myeloid leukemia (AML), chronic myeloid leukemia (CML), acute lymphoblastic leukemia (ALL), B-cell lineage acute lymphoblastic leukemia (B-ALL), T-cell lineage acute lymphoblastic leukemia (T-ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, eosinophilic leukemia, B myelomonocytic leukemia and Hodgkin's leukemia (HL).

15. The method of claim 14, wherein the cancer is acute myeloid leukemia (AML).

16. The method of claim 15, wherein the acute myeloid leukemia (AML) is refractory or relapse acute myeloid leukemia.

17. The method of claim 14, wherein the subject is a fit AML subject.

18. The method of claim 14, wherein the subject is an unfit AML subject.

21. The method of claim 14, wherein the acute myeloid leukemia (AML) is characterized by overexpression of P-glycoprotein, overexpression of EVI1, a p53 alteration, DNMT3A mutation, FLT3 internal tandem duplication, a complex karyotype, decreased expression in BRCA1, BRCA2, or PALB2, or mutations in BRCA1, BRCA2, or PALB2.

26. The method of claim 1, wherein a total daily dose of 20-3000 mg/m² of cytarabine is administered to the subject.

27. The method of claim 26, wherein cytarabine is administered to the subject daily or every other day.

28-29. (canceled)

30. The method of claim 1, wherein: (i) a total daily dose of 110 mg/m² of cytarabine is administered to the subject every day for 7 days, (ii) a total daily dose of 3000 mg/m² of cytarabine is administered to the subject every other day for 5 days, (iii) a total daily dose of 20 mg/m² of cytarabine is administered to the subject every day for 10 days, or (iv) a total daily dose of 200 mg/m² of cytarabine is administered to the subject every day for 7 days.

31-34. (canceled)

35. A pharmaceutical composition comprising: i) an effective amount of cytarabine; ii) an effective amount of an antibody-drug conjugate of Formula (I): 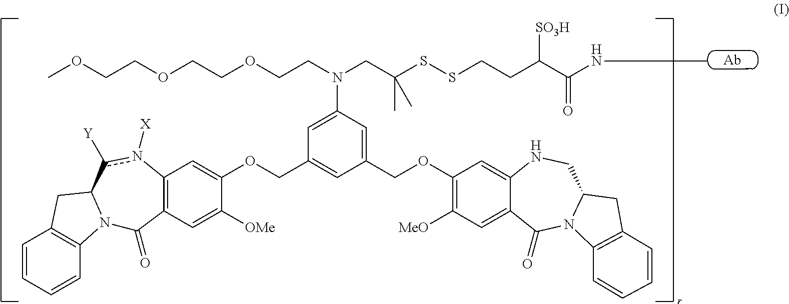
or a pharmaceutically acceptable salt thereof; and iii) a pharmaceutically acceptable carrier or diluent; wherein:
the double line [symbol] between N and C represents a single bond or a double bond, provided that when it is a double bond, X is absent and Y is hydrogen, when it is a single bond, X is hydrogen, Y is —SO₃H;
Ab is an anti-CD33 antibody or antigen-binding fragment thereof comprising a heavy chain variable region (V_{H}) complementary determining region (CDR)1 sequence of SEQ ID NO:1, a V_{H} CDR2 sequence of SEQ ID NO:2, and a V_{H} CDR3 sequence of SEQ ID NO:3, and a light chain variable region (V_{L}) CDR1 sequence of SEQ ID NO:4, a V_{L} CDR2 sequence of SEQ ID NO:5, and a V_{L} CDR3 sequence of SEQ ID NO:6; and
r is an integer from 1 to 10.

36-46. (canceled)
